(19) United States
(12) Patent Application Publication
(10) Pub. No.: US 2018/0346963 A1
(43) Pub. Date: Dec. 6, 2018

(54) Preparation of Concatenated Polynucleotides

(71) Applicant: Counsyl, Inc., South San Francisco, CA (US)

(72) Inventors: Noah C. Welker, Half Moon Bay, CA (US); Clement S. Chu, San Francisco, CA (US)

(21) Appl. No.: 15/994,624

(22) Filed: May 31, 2018

Related U.S. Application Data
(60) Provisional application No. 62/513,878, filed on Jun. 1, 2017.
(60) Provisional application No. 62/561,065, filed on Sep. 20, 2017.

(51) Int. Cl. C12Q 1/6806; C12Q 1/6869

(52) U.S. Cl. CPC C12Q 1/6806; C12Q 1/6869

(57) ABSTRACT

Methods for preparing concatenated nucleic acid molecules are provided. The methods herein include adaptors with complementary sequences for preparation of concatenated nucleic acid molecules, and methods of sequencing such nucleic acids.

### CROSS-REFERENCE TO RELATED APPLICATIONS

This application claims the benefit of U.S. Provisional Application Nos. 62/513,878, filed on Jun. 1, 2017, and 62/561,065, filed on Sep. 20, 2017, both of which are incorporated herein by reference in their entireties.

### FIELD OF THE INVENTION

The present invention relates to methods and compositions for producing concatenated nucleic acids.

### BACKGROUND

Next-generation sequencing (NGS) allows small-scale, inexpensive genome sequencing with a current turnaround time measured in hours-days. Next generation sequencing of nucleic acids has greatly increased the rate of genomic sequencing, thereby bringing in a new era for medical diagnostics, forensics, metagenomics, and many other applications.

However, the information that can be obtained via some NGS platforms, such as the Illumina platform, are limited by the number of sequenceable molecules (clusters) present on a fixed surface area, for example, surface area of a flow cell, with one unique nucleic acid molecule sequenced at a particular position (cluster). Methods that would increase the number of unique nucleic acid molecules that may be sequenced per unit area would be highly desirable. Increasing the number of reads that may be obtained per position on a surface would be advantageous, greatly increasing the amount of sequence information that can be obtained per unit surface area of the cell, while conserving reagents and decreasing the amount of time needed to obtain such information. For many molecular applications that use NGS data to provide counting data of molecular events, increasing the number of reads (not necessarily base pairs) is the most salient sequencing metric. A workflow that could increase the number of unique molecular reads available from a single flowcell would increase throughput and/or reduce cost by allowing for more molecular counting events on a given surface area (flowcell).

### BRIEF SUMMARY OF THE INVENTION

Methods and compositions are provided for preparing concatenated nucleic acid molecules. In some embodiments, concatenated nucleic acid molecules that are prepared as described herein are used in a method for nucleic acid sequencing.

In one aspect, methods are provided for preparing concatenated nucleic acid molecules. In some embodiments, the method includes: (a) incorporating a first adaptor into at least one first nucleic acid molecule that includes a first nucleic acid sequence (e.g., a first test nucleic acid sequence from a subject) and incorporating a second adaptor into at least one second nucleic acid molecule that includes a second nucleic acid sequence (e.g., a second test nucleic acid sequence from a subject), wherein the first adaptor comprises a first 3′ adaptor nucleic acid sequence including a first extendible 3′ end and the second adaptor comprises a second 3′ adaptor nucleic acid sequence comprising a second extendible 3′ end, wherein the first and second 3′ adaptor nucleic acid sequences are capable of hybridizing to each other; and (b) hybridizing and extending the first and second 3′ adaptor nucleic acid sequences, thereby producing extension products that include concatenated nucleic acid molecules including: (i) at least one first nucleic acid sequence and the complement of at least one second nucleic acid sequence, separated by adaptor sequences; and (ii) at least one second nucleic acid sequence and the complement of at least one first nucleic acid sequence, separated by adaptor sequences.

In one embodiment, the method includes: hybridizing and extending first and second nucleic acid molecules, wherein the first nucleic acid molecule includes a first test nucleic acid sequence from a subject and a first adaptor that is not from the subject, and wherein the first adaptor includes a first 3′ adaptor nucleic acid sequence including a first extendible 3′ end, wherein the second nucleic acid molecule includes a second test nucleic acid sequence from a subject and a second adaptor that is not from the subject, and wherein the second adaptor includes a second 3′ adaptor nucleic acid sequence and includes a second extendible 3′ end, and wherein the first and second 3′ adaptor nucleic acid sequences are capable of hybridizing to each other.

In one embodiment, the first and second nucleic acid sequences include double stranded nucleic acid sequences (e.g., a first double stranded test nucleic acid sequences from a subject and a second double stranded test nucleic acid sequence from a subject) with first and second ends, and each of the first and second adaptors includes: (i) a double stranded region; (ii) the first or second 3′ adaptor nucleic acid sequence, respectively, including a single stranded nucleic acid sequence that includes the first or second extendible 3′ end, respectively; and (iii) a single stranded nucleic acid sequence including a 5′ end, wherein the double stranded region of the first adaptor is attached (e.g., ligated) to first and second ends of the first double stranded nucleic acid sequence and the double stranded region of the second adaptor is attached (e.g., ligated) to first and second ends of the second double stranded nucleic acid sequence, and wherein the 3′ single stranded nucleic acid sequences of the first and second adaptors are capable of hybridizing to each other. In some embodiments, more than two nucleic acid sequences are concatenated. In some embodiments, the 5′ single stranded sequence of the first and/or the second adaptor includes one or more sample index sequence(s). In some embodiments, the 5′ single stranded sequence of the first and/or the second adaptor includes a flow cell binding sequence at its 5′ end.

In one embodiment, the first and second nucleic acid sequences are single stranded (e.g., a first single stranded test nucleic acid sequences from a subject and a second single stranded test nucleic acid sequence from a subject), the first and second adaptors are single stranded, and the 3′ single stranded nucleic acid sequences of the first and second adaptors are capable of hybridizing to each other.

In some embodiments, a 5′ phosphate group is added to the first and second adaptors prior to incorporating the adaptors into the first and second nucleic acid molecules.

In some embodiments, one or more sample index sequence and/or a flow cell binding sequence is incorporated into the 5′ end of the first and/or second nucleic acid molecule.

In some embodiments, the first and second nucleic acid sequences (e.g., first and second test nucleic acid sequences from a subject) are amplified prior to incorporating the adaptors into the first and second nucleic acid molecules, and/or prior to hybridizing and extending the first and second 3′ adaptor nucleic acid sequences. For example, the amplification may include polymerase chain reaction (PCR) or a linear amplification method. In some embodiments, PCT includes nested, semi-nested, or hemi-nested PCR.

In some embodiments, incorporation of the adaptors into the first and second nucleic acid molecules includes ligation of a first adaptor to at least one first nucleic acid sequence and ligation of a second adaptor to at least one second nucleic acid sequence. In some embodiments, the ligation reaction mixture includes a macromolecular crowding agent, such as, for example, polyethylene glycol. In some embodiments, the ligated nucleic acid molecules are amplified, prior hybridization and extension of the first and second 3′ adaptor nucleic acid sequences. For example, the amplification may include PCR or a linear amplification method. In some embodiments, PCR includes nested, semi-nested, or hemi-nested PCR. In one embodiment, ligation of the first adaptors are ligated to the first nucleic acid sequences in a separate reaction mixture from ligation of the second adaptors to the second nucleic acid sequences. In another embodiment, ligation of the first adaptors are ligated to the first nucleic acid sequences in the same reaction mixture as ligation of the second adaptors to the second nucleic acid sequences, and ligation of the first adaptors is temporally separated from ligation of the second adaptors.

In some embodiments, incorporation of the adaptors into the first and second nucleic acid molecules includes an amplification reaction. For example, the amplification reaction may include PCR or a linear amplification method. In one embodiment, the amplification reaction includes PCR, and the first and second nucleic acid molecules are PCR amplicons. In some embodiments, PCR includes nested, semi-nested, or hemi-nested PCR.

In some embodiments, the extension products that include concatenated nucleic acid molecules are amplified. For example, the amplification may include PCR or a linear amplification method. In some embodiments, PCR includes nested, semi-nested, or hemi-nested PCR.

In some embodiments, the at least one first nucleic acid molecule (e.g., at least one first test nucleic acid sequence from a subject) includes a plurality of different first nucleic acid sequences and the at least one second nucleic acid molecule (e.g., at least one second test nucleic acid sequence from a subject) includes a plurality of different second nucleic acid sequences.

The plurality of first nucleic acid molecules may be all from the same subject or from a plurality of different subjects. The plurality of second nucleic acid molecules may be all from the same subject or from a plurality of different subjects.

In one embodiment, the first and second nucleic acid molecules are from the same subject. In another embodiment, the first and second nucleic molecules are from different subjects. In one embodiment, the first and second nucleic acid molecules are from the same species. In another embodiment, the first and second nucleic acid molecules are from different species.

In some embodiments, the first and/or second adaptors include a sample or source specific barcode sequence. In some embodiments, amplification of the first and/or second nucleic acid molecules or the extension products comprises primers that comprise a sample or source specific barcode sequence, thereby incorporating the molecular barcode sequence into the amplified first and/or second nucleic acid molecules or extension products.

In some embodiments, the first and/or second nucleic acid molecules include cell-free DNA. For example, the cell-free DNA may include cell-free tumor DNA or cell-free fetal DNA. In some embodiments, the first and/or second nucleic acid molecules include RNA or cDNA. In some embodiments, the first and/or second nucleic acid molecules are enriched from a nucleic acid library.

In some embodiments, the extension products that include concatenated nucleic acid molecules are rendered competent for sequencing. For example, the extension products may be made competent to hybridize to a flow cell. In some embodiments, the method includes immobilizing the extension products on the surface of a flow cell.

In another aspect, methods are provided for nucleic acid sequencing. The methods include preparing concatenated nucleic acid molecules according to any of the methods described herein, and sequencing the extension products (i.e., the extension products that include concatenated nucleic acid molecules) or amplified extension products.

In some embodiments, the method includes sequencing the first and second nucleic acid sequences or complements thereof in the extension products using primers that are complementary to adaptor sequences that are upstream of the first and second nucleic acid sequences in the extension product. In some embodiments, the concatenated nucleic acid molecules include one or more sample index sequence (e.g., one or more sample index sequence in the first and/or second adaptor or introduced via amplification), and the method further comprises sequencing at least one sample index sequence using a primer that is complementary to a sequence that is upstream of the sample index sequence.

In one embodiment, the concatenated nucleic acid molecules include a flow cell binding sequence at the 5′ end (e.g., a flow cell binding sequence at the 5′ end of the first and/or second adaptor or introduced via amplification), and the extension products (i.e., the extension products that include concatenated nucleic acid molecules) or amplified extension products are immobilized on the surface of a flow cell by hybridization of the flow cell binding sequences to complementary sequences on the flow cell.

In another aspect, a nucleic acid sequencing library is provided. The sequencing library includes a plurality of extension products (i.e., extension products that include concatenated nucleic acid molecules) or amplified extension products produced according to any of the methods described herein.

In another aspect, concatenated nucleic acid molecules, prepared by any of the methods described herein, are provided. For example, concatenated nucleic acid molecules that include at least one sample nucleic acid sequences and the complement of at least one other sample nucleic acid sequence, separated by an adaptor sequences that is not a sample nucleic acid sequence, are provided. In some embodiments, concatenated nucleic acid molecules are provided that include: (i) at least one first nucleic acid sequence and the complement of at least one second nucleic acid sequence, separated by a first adaptor sequence, and (ii) at least one second nucleic acid sequence and the complement of at least one first nucleic acid sequence, separated by a second adaptor sequence.

In another aspect, methods are provided for preparing concatenated nucleic acid molecules, including: (a) ligating a first adaptor to at least one first double stranded nucleic acid molecule that includes first and second ends, and ligating a second adaptor to at least one second double stranded nucleic acid molecule that includes first and second ends, thereby producing first and second adaptor ligated nucleic acid molecules, wherein each of the first and second adaptors includes a double stranded region, wherein the first adaptor is attached to first and second ends of the first double stranded nucleic acid molecule and the second adaptor is attached to first and second ends of the second double stranded nucleic acid molecule; (b) amplifying the first and second adaptor ligated nucleic acid molecules in separate reaction mixtures with first and second amplification primers, thereby producing first and second amplified adaptor ligated nucleic acid molecules, wherein one or both of the first and second amplification primers includes a terminal 5′ phosphate group or wherein a 5′ terminal phosphate group is added to one or both ends of the amplified adaptor ligated nucleic acid molecules (e.g., added enzymatically, for example, with a kinase enzyme, such as polynucleotide 5′-hydroxyl-kinase); (c) combining the first and second amplified adaptor ligated nucleic acid molecules; and (d) ligating the first and second amplified adaptor ligated nucleic acid molecules, thereby producing concatenated nucleic acid molecules. In an embodiment, the 5′ end of one primer is blocked, and the 5′ end of the other primer is selectively phosphorylated (e.g., added enzymatically, for example, with a kinase enzyme, such as polynucleotide 5′-hydroxyl-kinase).

In one embodiment, the first and/or second adaptors are double stranded. In another embodiment, the first and/or second adaptors further include, in addition to the double stranded region: (i) a single stranded nucleic acid sequence that includes a 3′ end; and (ii) a single stranded nucleic acid sequence that includes a 5′ end.

In one embodiment, step (d) includes blunt end ligation. In another embodiment, the amplified adaptor ligated nucleic acid molecules include a restriction endonuclease recognition sequence, wherein the restriction endonuclease produces cohesive ends with a 3′ or 5′ overhang sequence, and the method further includes digestion with the restriction endonuclease enzyme prior to step (d).

In another aspect, methods are provided for preparing concatenated nucleic acid molecules, including: (a) incorporating a first adaptor into at least one first nucleic acid molecule that includes a first nucleic acid sequence, and incorporating a second adaptor into at least one second nucleic acid molecule that includes a second nucleic acid sequence, wherein incorporating includes amplification, thereby producing first and second amplification products, wherein the first nucleic acid molecule is amplified with primers that hybridize to the first nucleic acid sequence, thereby producing the first amplification product, and wherein one or both of the primers include a terminal 5′ phosphate group or wherein a 5′ terminal phosphate group is added to one or both ends of the first amplification product; and wherein the second nucleic acid molecule is amplified with primers that hybridize to the second nucleic acid sequence, thereby producing the second amplification product, and wherein one or both of the primers includes a 5′ sequence include a 5′ terminal phosphate group or wherein a 5′ terminal phosphate group is added to one or both ends of the second amplification product (e.g., added enzymatically, for example, with a kinase enzyme, such as polynucleotide 5′-hydroxyl-kinase); (b) combining the first and second amplification products; and (c) ligating the first and second amplification products, thereby producing concatenated nucleic acid molecules. The primers may be tailed or non-tailed. In an embodiment, the 5′ end of one primer is blocked, and the 5′ end of the other primer is selectively phosphorylated (e.g., added enzymatically, for example, with a kinase enzyme, such as polynucleotide 5′-hydroxyl-kinase).

In one embodiment, step (c) includes blunt end ligation. In another embodiment, the first and second amplification products include a restriction endonuclease recognition sequence, wherein the restriction endonuclease produces cohesive ends with a 3′ or 5′ overhang sequence, and the method further comprises digestion with the restriction endonuclease prior to step (c).

### BRIEF DESCRIPTION OF THE DRAWINGS

FIGS. 1A-1B show embodiments of nucleic acid molecules prepared and immobilized on the surface of a sequencing flow cell using techniques that are known in the art (1A) and concatenated nucleic acid molecules as described herein (1B).

FIG. 2 shows one non-limiting embodiment of a workflow for preparing concatenated nucleic acid molecules as described herein using ligated adaptors.

FIG. 3 shows one non-limiting embodiment for preparing concatenated nucleic acid molecules as described herein using PCR amplification.

FIGS. 4A-4C shows results of nucleic acid concatenation and library preparation as described in Example 1. Y-shaped adapters including a P5 sequencing adapter and concatenation sequence A were ligated the A-tailed cfDNA (4A). In a separate reaction, Y-shaped adapters including the reverse complement of a P7 sequencing adapter and the reverse complement of concatenation sequence A were ligated to A-tailed cfDNA (4B). The resulting products were annealed and extended with a DNA polymerase to create a library of nucleic acid molecules consisting of two cfDNA fragments separated by the concatenation sequence and flanked by P5 and P7 sequencing adapters (4C).

FIG. 5 shows the total number of mapped reads, following removal of molecular duplicates, for maternal cfDNA samples sequenced using both concatenated nucleic acid molecules prepared as described herein (concat_seq) and a standard nucleic acid library preparation, as described in Example 1.

FIG. 6 shows a comparison of fetal DNA reads (the fetal fraction) between replicate samples (same samples as in FIG. 5) prepared with the “standard” library preparation and the library preparation using the method disclosed herein, as described in Example 1.

FIG. 7 shows one non-limiting embodiment of a workflow for preparing concatenated nucleic acid molecules as described herein using ligated adaptors to facilitate the concatenation of two nucleic acid molecules.

FIG. 8 shows one non-limiting embodiment for preparing concatenated nucleic acid molecules as described herein using PCR amplification to attach adaptors that facilitate the concatenation of two nucleic acid molecules.

### DETAILED DESCRIPTION

The invention provides concatenated nucleic acid molecules and methods of producing them. Concatenated nucleic acids may be used in sequencing applications, thereby increasing the amount of sequence information available per sequencing reaction. In particular, adaptors with complementary sequences are attached to the ends of nucleic acid sequences of interest or incorporated via primer extension (e.g., amplification such as polymerase chain extension), and the complementary adaptor sequences are hybridized and extended to produce concatenated nucleic acids.

In certain embodiments, the invention relates to methods for preparing nucleic acids for sequencing, in particular preparation of concatenated nucleic acid sequences to increase the amount of sequence information obtainable per unit area within a flow cell.

Unless defined otherwise herein, all technical and scientific terms used herein have the same meaning as commonly understood by one of ordinary skill in the art to which this invention belongs. Singleton, et al., Dictionary of Microbiology and Molecular Biology, second ed., John Wiley and Sons, New York (1994), and Hale & Markham, The Harper Collins Dictionary of Biology, Harper Perennial, NY (1991) provide one of skill with a general dictionary of many of the terms used in this invention. Any methods and materials similar or equivalent to those described herein can be used in the practice or testing of the present invention.

The practice of the present invention will employ, unless otherwise indicated, conventional techniques of molecular biology (including recombinant techniques), microbiology, cell biology, and biochemistry, which are within the skill of the art. Such techniques are explained fully in the literature, for example, Molecular Cloning: A Laboratory Manual, second edition (Sambrook et al., 1989); Oligonucleotide Synthesis (M. J. Gait, ed., 1984; Current Protocols in Molecular Biology (F. M. Ausubel et al., eds., 1994); PCR: The Polymerase Chain Reaction (Mullis et al., eds., 1994); and Gene Transfer and Expression: A Laboratory Manual (Kriegler, 1990).

Numeric ranges provided herein are inclusive of the numbers defining the range.

Unless otherwise indicated, nucleic acids are written left to right in 5′ to 3′ orientation; amino acid sequences are written left to right in amino to carboxy orientation, respectively.

### Definitions

“A,” “an” and “the” include plural references unless the context clearly dictates otherwise.

The term “adaptor” herein refers to a polynucleotide that is attached to or incorporated into a test or sample nucleic acid sequence or nucleic acid sequence of interest to facilitate a downstream application, such as, but not limited to, nucleic acid sequencing. The adaptor can be composed of two distinct oligonucleotide molecules that are base-paired with one another, i.e., complementary. Alternatively, the adaptor can be composed of a single oligonucleotide that includes one or more regions of complementarity, and one or more non-complementary regions. Alternatively, the adaptor can be a single stranded oligonucleotide.

In general, as used herein, a sequence element located “at the 3′ end” includes the 3′-most nucleotide of the oligonucleotide, and a sequence element located “at the 5′ end” includes the 5′-most nucleotide of the oligonucleotide.

An “extendible 3′ end” refers an oligonucleotide with a terminal 3′ nucleotide that may be extended, for example, by a polymerase enzyme, e.g., a 3′ nucleotide that contains a 3′ hydroxyl group.

As used herein, the term “barcode” (also termed single molecule identifier (SMI)) refers to a known nucleic acid sequence that allows some feature of a polynucleotide with which the barcode is associated to be identified. In some embodiments, the feature of the polynucleotide to be identified is the sample from which the polynucleotide is derived. In some embodiments, barcodes are about or at least about 3, 4, 5, 6, 7, 8, 9, 10, 11, 12, 13, 14, 15, or more nucleotides in length. In some embodiments, barcodes are shorter than 10, 9, 8, 7, 6, 5, or 4 nucleotides in length. In some embodiments, barcodes associated with some polynucleotides are of different lengths than barcodes associated with other polynucleotides. In general, barcodes are of sufficient length and include sequences that are sufficiently different to allow the identification of samples based on barcodes with which they are associated. In some embodiments, a barcode, and the sample source with which it is associated, can be identified accurately after the mutation, insertion, or deletion of one or more nucleotides in the barcode sequence, such as the mutation, insertion, or deletion of 1, 2, 3, 4, 5, 6, 7, 8, 9, 10, or more nucleotides. In some embodiments, each barcode in a plurality of barcodes differ from every other barcode in the plurality at least three nucleotide positions, such as at least 3, 4, 5, 6, 7, 8, 9, 10, or more nucleotide positions. A plurality of barcodes may be represented in a pool of samples, each sample including polynucleotides comprising one or more barcodes that differ from the barcodes contained in the polynucleotides derived from the other samples in the pool. Samples of polynucleotides including one or more barcodes can be pooled based on the barcode sequences to which they are joined, such that all four of the nucleotide bases A, G, C, and T are approximately evenly represented at one or more positions along each barcode in the pool (such as at 1, 2, 3, 4, 5, 6, 7, 8, or more positions, or all positions of the barcode).

A “sample barcode” refers to a nucleic acid sequence, e.g., an index sequence, that identifies a sample or source of a sample uniquely.

A “molecular barcode” refers to a nucleic acid sequence that identifies an individual nucleic acid molecule, e.g., the specific nucleic acid sequence of a molecule from a specific individual.

A “blocking group” is any modification that prevents extension of a 3′ end of an oligonucleotide, such as by a polymerase, a ligase, and/or other enzymes.

The term “base pair” or “bp” as used herein refers to a partnership (i.e., hydrogen bonded pairing) of adenine (A) with thymine (T), or of cytosine (C) with guanine (G) in a double stranded DNA molecule. In some embodiments, a base pair may include A paired with Uracil (U), for example, in a DNA/RNA duplex.

A “causal genetic variant” is a genetic variant for which there is statistical, biological, and/or functional evidence of association with a disease or trait.

In general, a “complement” of a given nucleic acid sequence is a sequence that is fully complementary to and hybridizable to the given sequence. In general, a first sequence that is hybridizable to a second sequence or set of second sequences is specifically or selectively hybridizable to the second sequence or set of second sequences, such that hybridization to the second sequence or set of second sequences is preferred (e.g., thermodynamically more stable under a given set of conditions, such as stringent conditions commonly used in the art) in comparison with hybridization with other sequences during a hybridization reaction.

The term “complementary” herein refers to the broad concept of sequence complementarity in duplex regions of a single polynucleotide strand or between two polynucleotide strands between pairs of nucleotides through base-pairing. It is known that an adenine nucleotide is capable of forming specific hydrogen bonds (“base pairing”) with a nucleotide, which is thymine or uracil. Similarly, it is known that a cytosine nucleotide is capable of base pairing with a guanine nucleotide. However, in certain circumstances, hydrogen bonds may also form between other pairs of bases, e.g., between adenine and cytosine, etc. “Essentially complementary” herein refers to sequence complementarity in duplex regions of a single polynucleotide strand or between two polynucleotide strands, for example, wherein the complementarity is less than 100% but is greater than 90%, and retains the stability of the duplex region.

The term “derived from” encompasses the terms “originated from,” “obtained from,” “obtainable from,” “isolated from,” and “created from,” and generally indicates that one specified material finds its origin in another specified material or has features that can be described with reference to the another specified material.

The term “duplex” herein refers to a region of complementarity that exists between two polynucleotide sequences. The term “duplex region” refers to the region of sequence complementarity that exists between two oligonucleotides or two portions of a single oligonucleotide.

The term “end-repaired DNA” herein refers to DNA that has been subjected to enzymatic reactions in vitro to blunt-end 5′- and/or 3′-overhangs. Blunt ends can be obtained by filling in missing bases for a strand in the 5′ to 3′ direction using a polymerase, and by removing 3′-overhangs using an exonuclease. For example, T4 polymerase and/or Klenow DNA polymerase may be used for DNA end repair.

The terms “first end” and “second end” when used in reference to a nucleic acid molecule, herein refers to ends of a linear nucleic acid molecule.

A “gene” refers to a DNA segment that is involved in producing a polypeptide and includes regions preceding and following the coding regions as well as intervening sequences (introns) between individual coding segments (exons).

Typically, “hybridizable” sequences share a degree of sequence complementarity over all or a portion of their respective lengths, such as 25%-100% complementarity, including at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity.

“Hybridization” and “annealing” refer to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may include two nucleic acid strands forming a duplex structure, three or more strands forming a multi-stranded complex, a single self-hybridizing strand, or any combination of these. A hybridization reaction may constitute a step in a more extensive process, such as the initiation of polymerase chain reaction (PCR), ligation reaction, sequencing reaction, or cleavage reaction, e.g., enzymatic cleavage of a polynucleotide by a ribozyme. A first nucleic acid sequence that can be stabilized via hydrogen bonding with the bases of the nucleotide residues of a second sequence is said to be “hybridizable” to the second sequence. In such a case, the second sequence can also be said to be hybridizable to the first sequence. The term “hybridized” refers to a polynucleotide in a complex that is stabilized via hydrogen bonding between the bases of the nucleotide residues.

When referring to immobilization or attachment of molecules (e.g., nucleic acids) to a solid support, the terms “immobilized” and “attached” are used interchangeably herein, and both terms are intended to encompass direct or indirect, covalent or non-covalent attachment, unless indicated otherwise. In some embodiments, covalent attachment may be preferred, but generally all that is required is that the molecules (e.g., nucleic acids) remain immobilized or attached to the support under the conditions in which it is intended to use the support, for example in nucleic acid amplification and/or sequencing applications.

The terms “isolated,” “purified,” “separated,” and “recovered” as used herein refer to a material (e.g., a protein, nucleic acid, or cell) that is removed from at least one component with which it is naturally associated, for example, at a concentration of at least 90% by weight, or at least 95% by weight, or at least 98% by weight of the sample in which it is contained. For example, these terms may refer to a material which is substantially or essentially free from components which normally accompany it as found in its native state, such as, for example, an intact biological system. An isolated nucleic acid molecule includes a nucleic acid molecule contained in cells that ordinarily express the nucleic acid molecule, but the nucleic acid molecule is present extrachromosomally or at a chromosomal location that is different from its natural chromosomal location.

The terms “joining” and “ligation” as used herein, with respect to two polynucleotides, such as an adapter oligonucleotide and a sample polynucleotide, refers to the covalent attachment of two separate polynucleotides to produce a single larger polynucleotide with a contiguous backbone.

The term “library” herein refers to a collection or plurality of template molecules, i.e., template DNA duplexes, which share common sequences at their 5′ ends and common sequences at their 3′ ends. Use of the term “library” to refer to a collection or plurality of template molecules should not be taken to imply that the templates making up the library are derived from a particular source, or that the “library” has a particular composition. By way of example, use of the term “library” should not be taken to imply that the individual templates within the library must be of different nucleotide sequence or that the templates must be related in terms of sequence and/or source.

The term “mutation” herein refers to a change introduced into a parental sequence, including, but not limited to, substitutions, insertions, deletions (including truncations). The consequences of a mutation include, but are not limited to, the creation of a new character, property, function, phenotype or trait not found in the protein encoded by the parental sequence.

The term “Next Generation Sequencing (NGS)” herein refers to sequencing methods that allow for massively parallel sequencing of clonally amplified and of single nucleic acid molecules during which a plurality, e.g., millions, of nucleic acid fragments from a single sample or from multiple different samples are sequenced in unison. Non-limiting examples of NGS include sequencing-by-synthesis, sequencing-by-ligation, real-time sequencing, and nanopore sequencing.

The term “nucleotide” herein refers to a monomeric unit of DNA or RNA consisting of a sugar moiety (pentose), a phosphate, and a nitrogenous heterocyclic base. The base is linked to the sugar moiety via the glycosidic carbon (1′ carbon of the pentose) and that combination of base and sugar is a nucleoside. When the nucleoside contains a phosphate group bonded to the 3′ or 5′ position of the pentose it is referred to as a nucleotide. A sequence of polymeric operatively linked nucleotides is typically referred to herein as a “base sequence” or “nucleotide sequence,” or nucleic acid or polynucleotide “strand,” and is represented herein by a formula whose left to right orientation is in the conventional direction of 5′-terminus to 3′-terminus, referring to the terminal 5′ phosphate group and the terminal 3′ hydroxyl group at the “5′” and “3′” ends of the polymeric sequence, respectively.

The term “nucleotide analog” herein refers to analogs of nucleoside triphosphates, e.g., (S)-Glycerol nucleoside triphosphates (gNTPs) of the common nucleobases: adenine, cytosine, guanine, uracil, and thymidine (Horhota et al., Organic Letters, 8:5345-5347 [2006]). Also encompassed are nucleoside tetraphosphate, nucleoside pentaphosphates and nucleoside hexaphosphates.

The term “operably linked” refers to a juxtaposition or arrangement of specified elements that allows them to perform in concert to bring about an effect. For example, a promoter is operably linked to a coding sequence if it controls the transcription of the coding sequence.

The term “polymerase” herein refers to an enzyme that catalyzes the polymerization of nucleotides (i.e., the polymerase activity). The term polymerase encompasses DNA polymerases, RNA polymerases, and reverse transcriptases. A “DNA polymerase” catalyzes the polymerization of deoxyribonucleotides. An “RNA polymerase” catalyzes the polymerization of ribonucleotides. A “reverse transcriptase” catalyzes the polymerization of deoxyribonucleotides that are complementary to an RNA template.

The terms “polynucleotide,” “nucleotide,” “nucleotide sequence,” “nucleic acid,” “nucleic acid molecule,” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. and single- or multi-stranded (e.g., single-stranded, double-stranded, triple-helical, etc.), which contain deoxyribonucleotides, ribonucleotides, and/or analogs or modified forms of deoxyribonucleotides or ribonucleotides, including modified nucleotides or bases or their analogs. Because the genetic code is degenerate, more than one codon may be used to encode a particular amino acid, and the present invention encompasses polynucleotides which encode a particular amino acid sequence. Any type of modified nucleotide or nucleotide analog may be used, so long as the polynucleotide retains the desired functionality under conditions of use, including modifications that increase nuclease resistance (e.g., deoxy, 2′-O-Me, phosphorothioates, etc.). Labels may also be incorporated for purposes of detection or capture, for example, radioactive or nonradioactive labels or anchors, e.g., biotin. The term polynucleotide also includes peptide nucleic acids (PNA). Polynucleotides may be naturally occurring or non-naturally occurring. Polynucleotides may contain RNA, DNA, or both, and/or modified forms and/or analogs thereof. A sequence of nucleotides may be interrupted by non-nucleotide components. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need and circular portions. The following are nonlimiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, cDNA, recombinant polynucleotides, branched polynucleotides, plasmids, vectors, isolated DNA of any sequence, isolated RNA of any sequence, nucleic acid probes, adapters, and primers. A polynucleotide may include modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component, tag, reactive moiety, or binding partner. Polynucleotide sequences, when provided, are listed in the 5′ to 3′ direction, unless stated otherwise.

As used herein, “polypeptide” refers to a composition comprised of amino acids and recognized as a protein by those of skill in the art. The conventional one-letter or three-letter code for amino acid residues is used herein. The terms “polypeptide” and “protein” are used interchangeably herein to refer to polymers of amino acids of any length. The polymer may be linear or branched, it may include modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The term “primer” herein refers to an oligonucleotide, whether occurring naturally or produced synthetically, which is capable of acting as a point of initiation of nucleic acid synthesis when placed under conditions in which synthesis of a primer extension product which is complementary to a nucleic acid strand is induced, e.g., in the presence of four different nucleotide triphosphates and a polymerase enzyme, e.g., a thermostable enzyme, in an appropriate buffer (“buffer” includes pH, ionic strength, cofactors, etc.) and at a suitable temperature. The primer is preferably single-stranded for maximum efficiency in amplification, but may alternatively be double-stranded. If double-stranded, the primer is first treated to separate its strands before being used to prepare extension products. Preferably, the primer is an oligodeoxyribonucleotide. The primer must be sufficiently long to prime the synthesis of extension products in the presence of the polymerase, e.g., thermostable polymerase enzyme. The exact lengths of a primer will depend on many factors, including temperature, source of primer and use of the method. For example, depending on the complexity of the sequence of interest, the oligonucleotide primer typically contains 15-25 nucleotides, although it may contain more or few nucleotides. Short primer molecules generally require colder temperatures to form sufficiently stable hybrid complexes with template.

A “promoter” refers to a regulatory sequence that is involved in binding RNA polymerase to initiate transcription of a gene. A promoter may be an inducible promoter or a constitutive promoter. An “inducible promoter” is a promoter that is active under environmental or developmental regulatory conditions.

The term “sequencing library” herein refers to DNA that is processed for sequencing, e.g., using massively parallel methods, e.g., NGS. The DNA may optionally be amplified to obtain a population of multiple copies of processed DNA, which can be sequenced by NGS.

The term “single stranded overhang” or “overhang” is used herein to refer to a strand of a double stranded (ds) nucleic acid molecule that extends beyond the terminus of the complementary strand of the ds nucleic acid molecule. The term “5′ overhang” or “5′ overhanging sequence” is used herein to refer to a strand of a ds nucleic acid molecule that extends in a 5′ direction beyond the 3′ terminus of the complementary strand of the ds nucleic acid molecule. The term “3′ overhang” or “3′ overhanging sequence” is used herein to refer to a strand of a ds nucleic acid molecule that extends in a 3′ direction beyond the 5′ terminus of the complementary strand of the ds nucleic acid molecule.

A “spacer” may consist of a repeated single nucleotide (e.g., 1, 2, 3, 4, 5, 6, 7, 8, 9, 10, or more of the same nucleotide in a row), or a sequence of 2, 3, 4, 5, 6, 7, 8, 9, 10, or more nucleotides repeated 1, 2, 3, 4, 5, 6, 7, 8, 9, 10, or more times. A spacer may comprise or consist of a specific sequence, such as a sequence that does not hybridize to any sequence of interest in a sample. A spacer may comprise or consist of a sequence of randomly selected nucleotides.

A “subject” or “individual” refers to the source from which a biological sample is obtained, for example, but not limited to, a mammal (e.g., a human), an animal, a plant, or a microorganism (e.g., bacteria, fungi).

The phrases “substantially similar” and “substantially identical” in the context of at least two nucleic acids typically means that a polynucleotide includes a sequence that has at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% sequence identity, in comparison with a reference (e.g., wild-type) polynucleotide or polypeptide. Sequence identity may be determined using known programs such as BLAST, ALIGN, and CLUSTAL using standard parameters. (See, e.g., Altshul et al. (1990) J. Mol. Biol. 215:403-410; Henikoff et al. (1989) Proc. Natl. Acad. Sci. 89:10915; Karin et al. (1993) Proc. Natl. Acad. Sci. 90:5873; and Higgins et al. (1988) Gene 73:237). Software for performing BLAST analyses is publicly available through the National Center for Biotechnology Information. Also, databases may be searched using FASTA (Person et al. (1988) Proc. Natl. Acad. Sci. 85:2444-2448.) In some embodiments, substantially identical nucleic acid molecules hybridize to each other under stringent conditions (e.g., within a range of medium to high stringency).

Nucleic acid “synthesis” herein refers to any in vitro method for making a new strand of polynucleotide or elongating an existing polynucleotide (i.e., DNA or RNA) in a template dependent manner. Synthesis, according to the invention, can include amplification, which increases the number of copies of a polynucleotide template sequence with the use of a polymerase. Polynucleotide synthesis (e.g., amplification) results in the incorporation of nucleotides into a polynucleotide (e.g., extension from a primer), thereby forming a new polynucleotide molecule complementary to the polynucleotide template. The formed polynucleotide molecule and its template can be used as templates to synthesize additional polynucleotide molecules. “DNA synthesis,” as used herein, includes, but is not limited to, polymerase chain reaction (PCR), and may include the use of labeled nucleotides, e.g., for probes and oligonucleotide primers, or for polynucleotide sequencing.

The term “tag” refers to a detectable moiety that may be one or more atom(s) or molecule(s), or a collection of atoms and molecules. A tag may provide an optical, electrochemical, magnetic, or electrostatic (e.g., inductive, capacitive) signature.

The term “tagged nucleotide” herein refers to a nucleotide that includes a tag (or tag species) that is coupled to any location of the nucleotide including, but not limited to a phosphate (e.g., terminal phosphate), sugar or nitrogenous base moiety of the nucleotide. Tags may be one or more atom(s) or molecule(s), or a collection of atoms and molecules. A tag may provide an optical, electrochemical, magnetic, or electrostatic (e.g., inductive, capacitive) signature.

The term “DNA duplex” herein refers to a double stranded DNA molecule that is derived from a sample polynucleotide that is DNA, e.g., genomic or cell-free DNA (“cfDNA”), and/or RNA.

As used herein, the term “target polynucleotide” refers to a nucleic acid molecule or polynucleotide in a population of nucleic acid molecules having a target sequence to which one or more oligonucleotides are designed to hybridize. In some embodiments, a target sequence uniquely identifies a sequence derived from a sample, such as a particular genomic, mitochondrial, bacterial, viral, or RNA (e.g., mRNA, miRNA, primary miRNA, or pre-miRNA) sequence. In some embodiments, a target sequence is a common sequence shared by multiple different target polynucleotides, such as a common adapter sequence joined to different target polynucleotides. “Target polynucleotide” may be used to refer to a double-stranded nucleic acid molecule that includes a target sequence on one or both strands, or a single-stranded nucleic acid molecule including a target sequence, and may be derived from any source of or process for isolating or generating nucleic acid molecules. A target polynucleotide may include one or more (e.g., 1, 2, 3, 4, 5, 6, 7, 8, 9, 10, or more) target sequences, which may be the same or different. In general, different target polynucleotides include different sequences, such as one or more different nucleotides or one or more different target sequences.

The term “template DNA molecule” herein refers to a strand of a nucleic acid from which a complementary nucleic acid strand is synthesized by a DNA polymerase, for example, in a primer extension reaction.

The term “template-dependent manner” refers to a process that involves the template dependent extension of a primer molecule (e.g., DNA synthesis by DNA polymerase). The term “template-dependent manner” typically refers to polynucleotide synthesis of RNA or DNA wherein the sequence of the newly synthesized strand of polynucleotide is dictated by the well-known rules of complementary base pairing (see, for example, Watson, J. D. et al., In: Molecular Biology of the Gene, 4th Ed., W. A. Benjamin, Inc., Menlo Park, Calif. (1987)).

“Nested” polymerase chain reaction (PCR) refers to a method of PCR in which two sequential PCR reactions are performed, with two sets of primers. This method is intended to minimize the amplification of non-specific PCR products. During this method, the first reaction is performed with flanking primers while the second reaction is performed with internal primers that hybridize to a region within the first PCR product.

“Semi-nested” or “Hemi-nested” PCR refers to a variation of “Nested” PCR wherein two sequential PCR reaction are performed with two sets of primers. During this method, the first reaction is performed with flanking primers, while the second reaction is performed with one flanking primer from the first reaction and a second internal primer that hybridizes to a region within the first PCR product.

### Sample Nucleic Acid Sequences

Sample nucleic acid sequences, also termed “test” nucleic acid sequences herein, such as specific nucleic acid sequences of interest or random nucleic acid sequences from a subject, are concatenated in methods as described herein. Sample nucleic acid sequences are derived from a subject, e.g., derived from a biological sample from a subject. The nucleic acid sequences of interest may be double stranded or single stranded, or may include a combination of double stranded and single stranded regions.

Sample polynucleotides that can be used as the source for preparation of concatenated nucleic acid molecules as described herein include genomic cellular DNA, cell-free DNA, mitochondrial DNA, RNA, and cDNA.

In some embodiments, samples include DNA. In some embodiments, samples include genomic DNA. In some embodiments, samples include mitochondrial DNA, chloroplast DNA, plasmid DNA, bacterial artificial chromosomes, yeast artificial chromosomes, oligonucleotide tags, or combinations thereof. In some embodiments, the samples include DNA generated by amplification, such as by primer extension reactions using any suitable combination of primers and a DNA polymerase, including but not limited to polymerase chain reaction (PCR), reverse transcription, and combinations thereof. Where the template for the primer extension reaction is RNA, the product of reverse transcription is referred to as complementary DNA (cDNA). Primers useful in primer extension reactions can include sequences specific to one or more nucleic acid sequences of interest, random sequences, partially random sequences, and combinations thereof. Reaction conditions suitable for primer extension reactions are known in the art. In general, sample polynucleotides include any polynucleotide present in a sample, which may or may not include a polynucleotide sequence of interest. In some embodiments, a sample from a single individual is divided into multiple separate samples (e.g. 2, 3, 4, 5, 6, 7, 8, 9, 10, or more separate samples) that are subjected to the methods described herein independently, such as analysis in duplicate, triplicate, quadruplicate, or more.

In some embodiments, sample nucleic acid duplex molecules are provided, and are used to produce concatenated nucleic acid molecules in methods described herein. The nucleic acid duplex may be derived from a source in which it exists as double-stranded DNA, such as genomic DNA, or it may be prepared from a single-stranded nucleic acid source, such as RNA, e.g., cDNA.

### Biological Sample Sources

In some embodiments, a sample that includes genomic nucleic acids to which the methods described herein may be applied may a biological sample such as a tissue sample, a biological fluid sample, or a cell sample, and processed fractions thereof. The subject from which the sample is obtained may be a mammal, for example, a human. A biological fluid sample includes, as non-limiting examples, blood, plasma, serum, sweat, tears, sputum, urine, ear flow, lymph, interstitial fluid, saliva, cerebrospinal fluid, ravages, bone marrow suspension, vaginal flow, transcervical lavage, brain fluid, ascites, milk, secretions of the respiratory, intestinal and genitourinary tracts, amniotic fluid and leukophoresis samples. In some embodiments, the source sample is a sample that is easily obtainable by non-invasive procedures, e.g., blood, plasma, serum, sweat, tears, sputum, urine, ear flow, or saliva. In some embodiments, the biological sample is a peripheral blood sample, or the plasma and serum fractions. In other embodiments, the biological sample is a swab or smear, a biopsy specimen, or a cell culture. In another embodiment, the sample is a mixture of two or more biological samples, e.g., a biological sample comprising two or more of a biological fluid sample, a tissue sample, and a cell culture sample. As used herein, the terms “blood,” “plasma” and “serum” expressly encompass fractions or processed portions thereof. Similarly, where a sample is taken from a biopsy, swab, smear, etc., the “sample” expressly encompasses a processed fraction or portion derived from the biopsy, swab, smear, etc.

In some embodiments, biological samples can be obtained from sources, including, but not limited to, samples from different individuals, different developmental stages of the same or different individuals, different diseased individuals (e.g., individuals with cancer or suspected of having a genetic disorder), normal individuals, samples obtained at different stages of a disease in an individual, samples obtained from an individual subjected to different treatments for a disease, samples from individuals subjected to different environmental factors, or individuals with predisposition to a pathology, individuals with exposure to a pathogen such as an infectious disease agent (e.g., HIV), and individuals who are recipients of donor cells, tissues and/or organs. In some embodiments, the sample is a sample that includes a mixture of different source samples derived from the same or different subjects. For example, a sample can include a mixture of cells derived from two or more individuals, as is often found at crime scenes. In one embodiment, the sample is a maternal sample that is obtained from a pregnant female, for example a pregnant human woman. In this instance, the sample can be analyzed to provide a prenatal diagnosis of potential fetal disorders. Unless otherwise specified, a maternal sample includes a mixture of fetal and maternal DNA, e.g., cfDNA. In some embodiments, the maternal sample is a biological fluid sample, e.g., a blood sample. In other embodiments, the maternal sample is a purified cfDNA sample.

A sample can be an unprocessed biological sample, e.g., a whole blood sample. A source sample can be a partially processed biological sample, e.g., a blood sample that has been fractionated to provide a substantially cell-free plasma fraction. A source sample can be a biological sample containing purified nucleic acids, e.g., a sample of purified cfDNA derived from an essentially cell-free plasma sample. Processing of the samples can include freezing samples, e.g., tissue biopsy samples, fixing samples e.g. formalin-fixing, and embedding samples, e.g., paraffin-embedding. Partial processing of samples includes sample fractionation, e.g., obtaining plasma fractions from blood samples, and other processing steps required for analyses of samples collected during routine clinical work, in the context of clinical trials, and/or scientific research. Additional processing steps can include steps for isolating and purifying sample nucleic acids. Further processing of purified samples includes, for example, steps for the requisite modification of sample nucleic acids in preparation for sequencing. Preferably, the sample is an unprocessed or a partially processed sample.

Samples can also be obtained from in vitro cultured tissues, cells, or other polynucleotide-containing sources. The cultured samples can be taken from sources including, but not limited to, cultures (e.g., tissue or cells) maintained in different media and/or conditions (e.g., pH, pressure, or temperature), maintained for different periods of time, and/or treated with different factors or reagents (e.g., a drug candidate, or a modulator), or mixed cultures of different types of tissue or cells.

Biological samples can be obtained from a variety of subjects, including but not limited to, mammals, e.g., humans, and other organisms, including, plants, or cells from the subjects, or microorganisms (e.g., bacteria, fungi).

Biological samples from which the sample polynucleotides are derived can include multiple samples from the same individual, samples from different individuals, or combinations thereof. In some embodiments, a sample includes a plurality of polynucleotides from a single individual. In some embodiments, a sample includes a plurality of polynucleotides from two or more individuals. An individual is any organism or portion thereof from which sample polynucleotides can be derived, non-limiting examples of which include plants, animals, fungi, protists, monerans, viruses, mitochondria, and chloroplasts. Sample polynucleotides can be isolated from a subject, such as a cell sample, tissue sample, fluid sample, or organ sample derived therefrom (or cell cultures derived from any of these), including, for example, cultured cell lines, biopsy, blood sample, cheek swab, or fluid sample containing a cell (e.g., saliva). The subject may be an animal, including but not limited to, a cow, a pig, a mouse, a rat, a chicken, a cat, a dog, etc., and in some embodiments is a mammal, such as a human.

### Preparation of Sample Nucleic Acids

Methods for the extraction and purification of nucleic acids are well known in the art. For example, nucleic acids can be purified by organic extraction with phenol, phenol/chloroform/isoamyl alcohol, or similar formulations, including TRIzol and TriReagent. Other non-limiting examples of extraction techniques include: (1) organic extraction followed by ethanol precipitation, e.g., using a phenol/chloroform organic reagent, with or without the use of an automated nucleic acid extractor; (2) stationary phase adsorption; and (3) salt-induced nucleic acid precipitation methods, such precipitation methods being typically referred to as “salting-out” methods.

Another example of nucleic acid isolation and/or purification includes the use of magnetic particles to which nucleic acids can specifically or non-specifically bind, followed by isolation of the beads using a magnet, and washing and eluting the nucleic acids from the beads. In some embodiments, the above isolation methods may be preceded by an enzyme digestion step to help eliminate unwanted protein from the sample, e.g., digestion with proteinase K, or other like proteases. If desired, RNase inhibitors may be added to the lysis buffer.

For certain cell or sample types, it may be desirable to add a protein denaturation/digestion step to the protocol. Purification methods may be directed to isolate DNA, RNA, or both. When both DNA and RNA are isolated together during or subsequent to an extraction procedure, further steps may be employed to purify one or both separately from the other. Sub-fractions of extracted nucleic acids can also be generated, for example, purification by size, sequence, or other physical or chemical characteristic.

In addition to an initial nucleic acid isolation step, purification of nucleic acids can be performed after any step in the methods described herein, such as to remove excess or unwanted reagents, reactants, or products. Methods for determining the amount and/or purity of nucleic acids in a sample are known in the art, and include absorbance (e.g., absorbance of light at 260 nm, 280 nm, and a ratio of these) and detection of a label (e.g., fluorescent dyes and intercalating agents, such as SYBR green, SYBR blue, DAPI, propidium iodine, Hoechst stain, SYBR gold, ethidium bromide).

In some embodiments, sample nucleic acid molecules are fragmented, e.g., fragmentation of cellular genomic DNA. Fragmentation of polynucleotide molecules by mechanical means cleaves the DNA backbone at C—O, P—O and C—C bonds, resulting in a heterogeneous mix of blunt and 3′- and 5′-overhanging ends with broken C—O, P—O and/C—C bonds (Alnemri and Litwack (1990) J Biol Chem 265:17323-17333; Richards and Boyer (1965) J Mol Biol 11:327-340), which may need to be repaired for subsequent method steps. Therefore, fragmentation of polynucleotides, e.g., cellular genomic DNA, may be required. Alternatively, fragmentation of cfDNA, which exists as fragments of <300 bases, may not necessary.

In some embodiments, polynucleotides are fragmented into a population of fragmented polynucleotides of one or more specific size range(s). In some embodiments, the amount of sample polynucleotides subjected to fragmentation is about, less than about, or more than about 50 ng, 100 ng, 200 ng, 300 ng, 400 ng, 500 ng, 600 ng, 700 ng, 800 ng, 900 ng, 1000 ng, 1500 ng, 2000 ng, 2500 ng, 5000 ng, 1 μg, 10 μg, or more. In some embodiments, fragments are generated from about, less than about, or more than about 1, 10, 100, 1000, 10,000, 100,000, 300,000, 500,000, or more genome-equivalents of starting DNA. In some embodiments, the fragments have an average or median length from about 10 to about 10,000 nucleotides (e.g., base pairs). In some embodiments, the fragments have an average or median length from about 50 to about 2,000 nucleotides (e.g., base pairs). In some embodiments, the fragments have an average or median length of about, less than about, more than about, or about 100 to about 2500, about 200 to about 1000, about 10 to about 800, about 10 to about 500, about 50 to about 500, about 50 to about 250, or about 50 to about 150 nucleotides (e.g., base pairs). In some embodiments, the fragments have an average or median length of about 300 to about 800 nucleotides (e.g., base pairs). In some embodiments, the fragments have an average or median length of about, less than about, or more than about 200, 300, 500, 600, 800, 1000, 1500 or more nucleotides (e.g., base pairs).

Fragmentation may be accomplished by methods known in the art, including chemical, enzymatic, and mechanical fragmentation. In some embodiments, the fragmentation is accomplished mechanically, including subjecting sample polynucleotides to acoustic sonication. In some embodiments, the fragmentation includes treating the sample polynucleotides with one or more enzymes under conditions suitable for the one or more enzymes to generate double-stranded nucleic acid breaks. Examples of enzymes useful in the generation of polynucleotide fragments include sequence specific and non-sequence specific nucleases. Non-limiting examples of nucleases include DNase I, Fragmentase, restriction endonucleases, variants thereof, and combinations thereof. For example, digestion with DNase I can induce random double-stranded breaks in DNA in the absence of Mg²⁺ and in the presence of Mn²⁺.

In some embodiments, fragmentation includes treating the sample polynucleotides with one or more restriction endonucleases. Fragmentation can produce fragments having 5′ overhangs, 3′ overhangs, blunt ends, or a combination thereof. In some embodiments, such as when fragmentation includes the use of one or more restriction endonucleases, cleavage of sample polynucleotides leaves overhangs having a predictable sequence.

In some embodiments, the method includes the step of size selecting the fragments via standard methods such as column purification or isolation from an agarose gel. In some embodiments, the method includes determining the average and/or median fragment length after fragmentation. In some embodiments, samples having an average and/or median fragment length above a desired threshold are again subjected to fragmentation. In some embodiments, samples having an average and/or median fragment length below a desired threshold are discarded.

In some embodiments, the 5′ and/or 3′ end nucleotide sequences of fragmented polynucleotides are not modified prior to incorporation (e.g., ligation) of adapters.

Polynucleotide fragments having an overhang can be joined to one or more adapters having a complementary overhang, such as in a ligation reaction. For example, fragmentation by a restriction endonuclease can be used to leave a predictable overhang, followed by joining (e.g., ligation) with an adapter having an overhang sequence that is complementary to the predictable overhang on a polynucleotide fragment.

In another example, cleavage by an enzyme that leaves a predictable blunt end can be followed by ligation of blunt-ended polynucleotide fragments to adapters that include a blunt end sequence. In some embodiments, the fragmented polynucleotides are blunt-end polished (or “end repaired”) to produce polynucleotide fragments having blunt ends, prior to being joined to adapters.

In an embodiment, a single adenine can be added to the 3′ ends of end repaired polynucleotide fragments using a template independent polymerase, followed by joining (e.g., ligation) to one or more adapters each having an overhanging thymine at a 3′ end.

In some embodiments, adapters can be joined to blunt end double-stranded DNA fragment molecules which have been modified by extension of the 3′ end with one or more nucleotides followed by 5′ phosphorylation. In some cases, extension of the 3′ end may be performed with a polymerase such as for example Klenow polymerase or any other suitable polymerases known in the art, or by use of a terminal deoxynucleotide transferase, in the presence of one or more dNTPs in a suitable buffer containing magnesium. In some embodiments, sample polynucleotides having blunt ends are joined to adapters having a blunt end.

Phosphorylation of 5′ ends of fragmented polynucleotides may be performed, for example, with T4 polynucleotide kinase in a suitable buffer containing ATP and magnesium.

Fragmented polynucleotides may optionally be treated to dephosphorylate 5′ ends or 3′ ends, for example, by using enzymes known in the art, such as phosphatases.

### Nucleic Acid Sequences of Interest

In some embodiments, the sample nucleic acid includes a variant sequence, e.g., a causal genetic variant or an aneuploidy. A single causal genetic variant can be associated with more than one disease or trait. In some embodiments, a causal genetic variant can be associated with a Mendelian trait, a non-Mendelian trait, or both. Causal genetic variants can manifest as variations in a polynucleotide, such as at least about 1, 2, 3, 4, 5, 6, 7, 8, 9, 10, 20, 50, or more sequence differences (such as between a polynucleotide including the causal genetic variant and a polynucleotide lacking the causal genetic variant at the same relative genomic position).

Non-limiting examples of types of causal genetic variants include single nucleotide polymorphisms (SNP), deletion/insertion polymorphisms (DIP), copy number variants (CNV), short tandem repeats (STR), restriction fragment length polymorphisms (RFLP), simple sequence repeats (SSR), variable number of tandem repeats (VNTR), randomly amplified polymorphic DNA (RAPD), amplified fragment length polymorphisms (AFLP), inter-retrotransposon amplified polymorphisms (IRAP), long and short interspersed elements (LINE/SINE), long tandem repeats (LTR), mobile elements, retrotransposon microsatellite amplified polymorphisms, retrotransposon-based insertion polymorphisms, sequence specific amplified polymorphism, and heritable epigenetic modification (for example, DNA methylation).

A causal genetic variant may also be a set of closely related causal genetic variants. Some causal genetic variants may exert influence as sequence variations in RNA polynucleotides. At this level, some causal genetic variants are also indicated by the presence or absence of a species of RNA polynucleotides. Also, some causal genetic variants result in sequence variations in protein polypeptides. A number of causal genetic variants are known in the art. An example of a causal genetic variant that is a SNP is the Hb S variant of hemoglobin that causes sickle cell anemia. An example of a causal genetic variant that is a DIP is the delta508 mutation of the CFTR gene which causes cystic fibrosis. An example of a causal genetic variant that is a CNV is trisomy 21, which causes Down's syndrome. An example of a causal genetic variant that is an STR is tandem repeat that causes Huntington's disease. Non-limiting examples of causal genetic variants are described in US2010/0022406, which is incorporated by reference in its entirety.

Causal genetic variants can be originally discovered by statistical and molecular genetic analyses of the genotypes and phenotypes of individuals, families, and populations. The causal genetic variants for Mendelian traits are typically identified in a two-stage process. In the first stage, families are identified in which multiple individuals who possess the trait are examined for genotype and phenotype. Genotype and phenotype data from these families is used to establish the statistical association between the presence of the Mendelian trait and the presence of a number of genetic markers. This association establishes a candidate region in which the causal genetic variant is likely to map. In a second stage, the causal genetic variant itself is identified. The second step typically entails sequencing the candidate region. More sophisticated, one-stage processes are possible with more advanced technologies which permit the direct identification of a causal genetic variant or the identification of smaller candidate regions. After one causal genetic variant for a trait is discovered, additional variants for the same trait can be discovered. For example, the gene associated with the trait can be sequenced in individuals who possess the trait or their relatives. Many causal genetic variants are cataloged in databases including the Online Mendelian Inheritance in Man (OMIM) and the Human Gene Mutation Database (HGMD).

A causal genetic variant may exist at any frequency within a specified population. In some embodiments, a causal genetic variant causes a trait having an incidence of no more than 1% a reference population. In another embodiment, a causal genetic variants causes a trait having an incidence of no more than 1/10,000 in a reference population.

In some embodiments, a causal genetic variant which is associated with a disease or trait is a genetic variant, the presence of which increases the risk of having or developing the disease or trait by about, less than about, or more than about 1%, 5%, 10%, 15%, 20%, 25%, 30%, 40%, 50%, 60%, 70%, 80%, 90%, 100%, 200%, 300%, 400%, 500%, or more. In some embodiments, a causal genetic variant is a genetic variant the presence of which increases the risk of having or developing a disease or trait by about, less than about, or more than about 1-fold, 2-fold, 3-fold, 4-fold, 5-fold, 6-fold, 7-fold, 8-fold, 9-fold, 10-fold, 25-fold, 50-fold, 100-fold, 500-fold, 1000-fold, 10000-fold, or more. In some embodiments, a causal genetic variant is a genetic variant the presence of which increases the risk of having or developing a disease or trait by any statistically significant amount, such as an increase having a p-value of about or less than about 0.1, 0.05, 10⁻³, 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, 10⁻¹⁵, or smaller.

In some embodiments, a causal genetic variant has a different degree of association with a disease or trait between two or more different populations of individuals, such as between two or more human populations. In some embodiments, a causal genetic variant has a statistically significant association with a disease or trait only within one or more populations, such as one or more human populations. A human population can be a group of people sharing a common genetic inheritance, such as an ethnic group. A human population can be a haplotype population or group of haplotype populations. A human population can be a national group. A human population can be a demographic population such as those delineated by age, gender, and socioeconomic factors. Human populations can be historical populations. A population can consist of individuals distributed over a large geographic area such that individuals at extremes of the distribution may never meet one another. The individuals of a population can be geographically dispersed into discontinuous areas. Populations can be informative about biogeographical ancestry. Populations can also be defined by ancestry. Genetic studies can define populations. In some embodiments, a population may be based on ancestry and genetics. A sub-population may serve as a population for the purpose of identifying a causal genetic variant.

In some embodiments, a causal genetic variant is associated with a disease, such as a rare genetic disease. Examples of rare genetic diseases include, but are not limited to: 21-Hydroxylase Deficiency, ABCC8-Related Hyperinsulinism, ARSACS, Achondroplasia, Achromatopsia, Adenosine Monophosphate Deaminase 1, Agenesis of Corpus Callosum with Neuronopathy, Alkaptonuria, Alpha-1-Antitrypsin Deficiency, Alpha-Mannosidosis, Alpha-Sarcoglycanopathy, Alpha-Thalassemia, Alzheimers, Angiotensin II Receptor, Type I, Apolipoprotein E Genotyping, Argininosuccinicaciduria, Aspartylglycosaminuria, Ataxia with Vitamin E Deficiency, Ataxia-Telangiectasia, Autoimmune Polyendocrinopathy Syndrome Type 1, BRCA1 Hereditary Breast/Ovarian Cancer, BRCA2 Hereditary Breast/Ovarian Cancer, Bardet-Biedl Syndrome, Best Vitelliform Macular Dystrophy, Beta-Sarcoglycanopathy, Beta-Thalassemia, Biotinidase Deficiency, Blau Syndrome, Bloom Syndrome, CFTR-Related Disorders, CLN3-Related Neuronal Ceroid-Lipofuscinosis, CLN5-Related Neuronal Ceroid-Lipofuscinosis, CLN8-Related Neuronal Ceroid-Lipofuscinosis, Canavan Disease, Carnitine Palmitoyltransferase IA Deficiency, Carnitine Palmitoyltransferase II Deficiency, Cartilage-Hair Hypoplasia, Cerebral Cavernous Malformation, Choroideremia, Cohen Syndrome, Congenital Cataracts, Facial Dysmorphism, and Neuropathy, Congenital Disorder of Glycosylationla, Congenital Disorder of Glycosylation Ib, Congenital Finnish Nephrosis, Crohn Disease, Cystinosis, DFNA 9 (COCH), Diabetes and Hearing Loss, Early-Onset Primary Dystonia (DYTI), Epidermolysis Bullosa Junctional, Herlitz-Pearson Type, FANCC-Related Fanconi Anemia, FGFR1-Related Craniosynostosis, FGFR2-Related Craniosynostosis, FGFR3-Related Craniosynostosis, Factor V Leiden Thrombophilia, Factor V R2 Mutation Thrombophilia, Factor XI Deficiency, Factor XIII Deficiency, Familial Adenomatous Polyposis, Familial Dysautonomia, Familial Hypercholesterolemia Type B, Familial Mediterranean Fever, Free Sialic Acid Storage Disorders, Frontotemporal Dementia with Parkinsonism-17, Fumarase deficiency, GJB2-Related DFNA 3 Nonsyndromic Hearing Loss and Deafness, GJB2-Related DFNB 1 Nonsyndromic Hearing Loss and Deafness, GNE-Related Myopathies, Galactosemia, Gaucher Disease, Glucose-6-Phosphate Dehydrogenase Deficiency, Glutaricacidemia Type 1, Glycogen Storage Disease Type 1a, Glycogen Storage Disease Type Ib, Glycogen Storage Disease Type II, Glycogen Storage Disease Type III, Glycogen Storage Disease Type V, Gracile Syndrome, HFE-Associated Hereditary Hemochromatosis, Halder AIMs, Hemoglobin S Beta-Thalassemia, Hereditary Fructose Intolerance, Hereditary Pancreatitis, Hereditary Thymine-Uraciluria, Hexosaminidase A Deficiency, Hidrotic Ectodermal Dysplasia 2, Homocystinuria Caused by Cystathionine Beta-Synthase Deficiency, Hyperkalemic Periodic Paralysis Type 1, Hyperornithinemia-Hyperammonemia-Homocitrullinuria Syndrome, Hyperoxaluria, Primary, Type 1, Hyperoxaluria, Primary, Type 2, Hypochondroplasia, Hypokalemic Periodic Paralysis Type 1, Hypokalemic Periodic Paralysis Type 2, Hypophosphatasia, Infantile Myopathy and Lactic Acidosis (Fatal and Non-Fatal Forms), Isovaleric Acidemias, Krabbe Disease, LGMD2I, Leber Hereditary Optic Neuropathy, Leigh Syndrome, French-Canadian Type, Long Chain 3-Hydroxyacyl-CoA Dehydrogenase Deficiency, MELAS, MERRF, MTHFR Deficiency, MTHFR Thermolabile Variant, MTRNR1-Related Hearing Loss and Deafness, MTTS1-Related Hearing Loss and Deafness, MYH-Associated Polyposis, Maple Syrup Urine Disease Type 1A, Maple Syrup Urine Disease Type 1B, McCune-Albright Syndrome, Medium Chain Acyl-Coenzyme A Dehydrogenase Deficiency, Megalencephalic Leukoencephalopathy with Subcortical Cysts, Metachromatic Leukodystrophy, Mitochondrial Cardiomyopathy, Mitochondrial DNA-Associated Leigh Syndrome and NARP, Mucolipidosis IV, Mucopolysaccharidosis Type I, Mucopolysaccharidosis Type IIIA, Mucopolysaccharidosis Type VII, Multiple Endocrine Neoplasia Type 2, Muscle-Eye-Brain Disease, Nemaline Myopathy, Neurological phenotype, Niemann-Pick Disease Due to Sphingomyelinase Deficiency, Niemann-Pick Disease Type C1, Nijmegen Breakage Syndrome, PPT1-Related Neuronal Ceroid-Lipofuscinosis, PROP1-related pituitary hormone deficiency, Pallister-Hall Syndrome, Paramyotonia Congenita, Pendred Syndrome, Peroxisomal Bifunctional Enzyme Deficiency, Pervasive Developmental Disorders, Phenylalanine Hydroxylase Deficiency, Plasminogen Activator Inhibitor I, Polycystic Kidney Disease, Autosomal Recessive, Prothrombin G20210A Thrombophilia, Pseudovitamin D Deficiency Rickets, Pycnodysostosis, Retinitis Pigmentosa, Autosomal Recessive, Bothnia Type, Rett Syndrome, Rhizomelic Chondrodysplasia Punctata Type 1, Short Chain Acyl-CoA Dehydrogenase Deficiency, Shwachman-Diamond Syndrome, Sjogren-Larsson Syndrome, Smith-Lemli-Opitz Syndrome, Spastic Paraplegia 13, Sulfate Transporter-Related Osteochondrodysplasia, TFR2-Related Hereditary Hemochromatosis, TPP1-Related Neuronal Ceroid-Lipofuscinosis, Thanatophoric Dysplasia, Transthyretin Amyloidosis, Trifunctional Protein Deficiency, Tyrosine Hydroxylase-Deficient DRD, Tyrosinemia Type I, Wilson Disease, X-Linked Juvenile Retinoschisis and Zellweger Syndrome Spectrum.

In some embodiments, the sample nucleic acid sequence includes a non-subject sequence. In general, a non-subject sequence corresponds to a polynucleotide derived from an organism other than the individual being tested, such as DNA or RNA from bacteria, archaea, viruses, protists, fungi, or other organism. A non-subject sequence may be indicative of the identity of an organism or class of organisms, and may further be indicative of a disease state, such as infection. An example of non-subject sequences useful in identifying an organism include, without limitation, ribosomal RNA (rRNA) sequences, such as 16s rRNA sequences (see, e.g., WO2010/151842). In some embodiments, non-subject sequences are analyzed instead of, or separately from causal genetic variants. In some embodiments, causal genetic variants and non-subject sequences are analyzed in parallel, such as in the same sample and/or in the same report.

### Adaptors

Polynucleotide adaptors are provided for use in the methods disclosed herein. Adaptors may be single stranded, double stranded, or partially double stranded (e.g., Y-shaped).

Adaptors as described herein include a 3′ nucleic acid sequence with an extendible 3′ end. First and second adaptors as described in the disclosed methods for preparing concatenated nucleic molecules have 3′ nucleic acid sequences that are capable of hybridizing to each other (e.g., complementary 3′ first and second adaptor sequences).

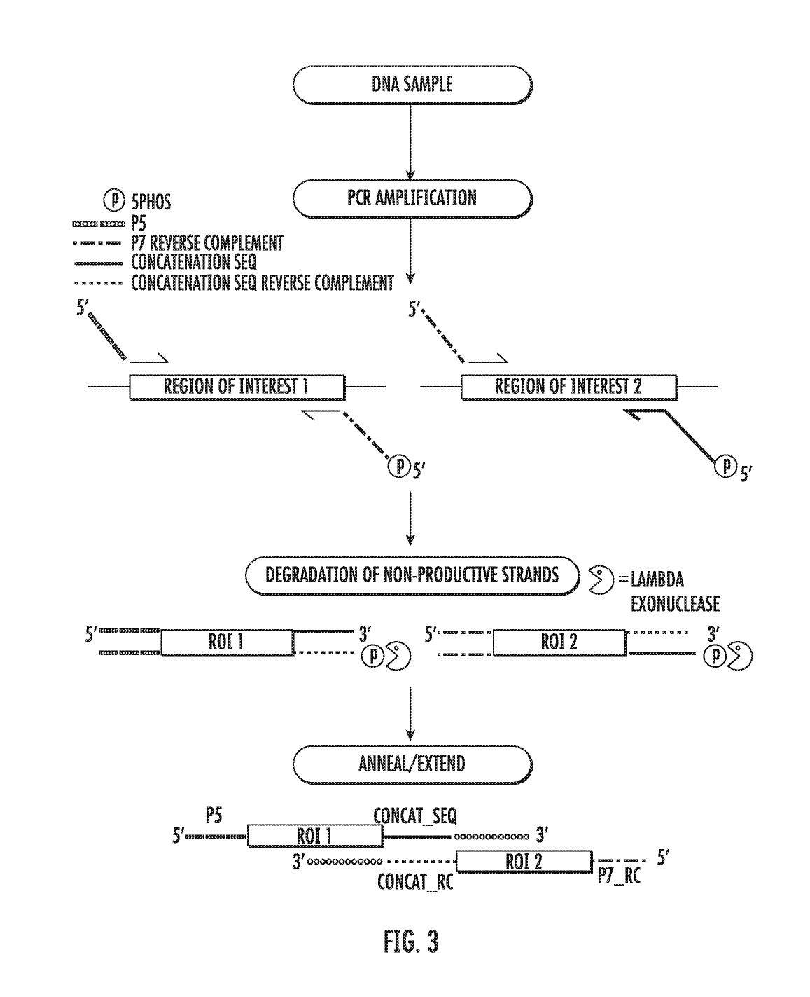

In some embodiments, adaptor sequences are introduced via an amplification reaction, such as PCR, using tailed primers. In one embodiment, concatenated nucleic acid molecules are prepared from PCR amplicons. Complementary extendible sequences 3′ to nucleic acid sequences of interest are introduced via the amplification reaction, a non-limiting example of which is depicted in FIG. 3.

In an embodiment, the nucleic acid molecules to be prepared for concatenation are double stranded, and the adaptors include: (i) a double stranded region; (ii) a first single stranded region that includes an extendible 3′ end; and (iii) a second single stranded region that includes a 5′ end. First adaptors are incorporated into (e.g., ligated to) each end of first nucleic acid duplexes (e.g., first adaptors incorporated into a plurality of different first nucleic acid duplexes) and second adaptors are incorporated into (e.g., ligated to) each end of second nucleic acid duplexes (e.g., second adaptors incorporated into a plurality of different second nucleic acid duplexes). The first single stranded region of the first adaptor includes an extendible 3′ nucleic acid sequence that is hybridizable (e.g., complementary) to a 3′ nucleic acid sequence in the first single stranded region of the second adaptor, such that they will anneal under appropriate conditions to join the first and second nucleic acid molecules together to form concatenated nucleic acid molecules. The 3′ ends can be extended to produce primer extension products, which may optionally be amplified prior to sequencing.

In another embodiment, the nucleic acid molecules to be prepared for concatenation are single stranded, and the adaptors are single stranded. First single stranded adaptors are incorporated into (e.g., ligated to) each end of first single stranded nucleic acid molecules (e.g., first adaptors incorporated into a plurality of different first single stranded nucleic acid molecules) and second single stranded adaptors are incorporated into (e.g., ligated to) each end of second single stranded nucleic acid molecules (e.g., second adaptors incorporated into a plurality of different second single stranded nucleic acid molecules). The first single stranded adaptor includes an extendible 3′ nucleic acid sequence that is hybridizable (e.g., complementary) to an extendible 3′ nucleic acid sequence of the second single stranded adaptor, such that they will anneal under appropriate conditions to join first and second single stranded nucleic acid molecules together to form concatenated nucleic acid molecules.

In another embodiment, the nucleic acid molecules to be prepared for concatenation are double stranded, and the adaptors are double stranded. First double stranded adaptors are incorporated into (e.g., ligated to) each end of first double stranded nucleic acid molecules (e.g., first adaptors incorporated into a plurality of different first double stranded nucleic acid molecules) and second double stranded adaptors are incorporated into (e.g., ligated to) each end of second double stranded nucleic acid molecules (e.g., second adaptors incorporated into a plurality of different second double stranded nucleic acid molecules). The first double stranded adaptor includes an extendible 3′ nucleic acid sequence that is hybridizable (e.g., complementary) to an extendible 3′ nucleic acid sequence of the second single stranded adaptor, such that they will anneal under appropriate conditions to join first and second single stranded nucleic acid molecules together to form concatenated nucleic acid molecules.

In some embodiments, adaptors are incorporated via amplification, for example, polymerase chain reaction (PCR) or a linear amplification method. In some embodiments, adaptors are in the form of tailed primers for amplification (e.g., PCR primers), and the adaptor sequences are incorporated by hybridization to a nucleic acid sequence of interest and extension via the amplification reaction. In one embodiment, the amplification reaction includes PCR amplification, and the nucleic acid products include the sequences of interest joined to adaptor (primer tail) sequences as PCR amplicons.

In some embodiments, adaptors include one or more nucleic acid sequences that are functional in a downstream application of use and that are incorporated into concatenated nucleic acid molecules produced as described herein. For example, an adaptor sequence that is incorporated into the concatenated nucleic acid molecule may include one or more sample index sequence(s) and/or a flow binding sequence.

In some embodiments, adaptors include one or more sample or source specific barcode sequence.

### Joining of Adaptors to Sample Nucleic Acid Molecules

Methods for joining two polynucleotides (e.g., adaptors and sample nucleic acids) are known in the art, and include without limitation, enzymatic (e.g., ligation with a ligase enzyme) and non-enzymatic (e.g., chemical) methods. Examples of polynucleotide joining reactions that are non-enzymatic include, for example, the non-enzymatic techniques described in U.S. Pat. Nos. 5,780,613 and 5,476,930, which are herein incorporated by reference.

In some embodiments, an adapter oligonucleotide is joined to a sample nucleic acid, e.g., a fragmented polynucleotide duplex, by a ligase, for example a DNA ligase or RNA ligase. Multiple ligases, each having characterized reaction conditions, are known in the art, and include, without limitation NAD⁺-dependent ligases including tRNA ligase, Taq DNA ligase, Thermus filiformis DNA ligase, Escherichia coli DNA ligase, Tth DNA ligase, Thermus scotoductus DNA ligase (I and II), thermostable ligase, Ampligase thermostable DNA ligase, VanC-type ligase, 9° N DNA Ligase, Tsp DNA ligase, and novel ligases discovered by bioprospecting; ATP-dependent ligases including T4 RNA ligase, T4 DNA ligase, T3 DNA ligase, T7 DNA ligase, Pfu DNA ligase, DNA ligase 1, DNA ligase III, DNA ligase IV, and novel ligases discovered by bioprospecting; and wild-type, mutant isoforms, and genetically engineered variants thereof.

Polynucleotide joining reactions (e.g., ligation) can be between polynucleotides having hybridizable sequences, such as complementary overhangs. Polynucleotide joining reactions (e.g., ligation) can also be between two blunt ends.

Generally, a 5′ phosphate is utilized in a ligation reaction. The 5′ phosphate can be provided by the fragmented polynucleotide, the adapter oligonucleotide, or both. 5′ phosphates can be added to or removed from polynucleotides to be joined, as needed. Methods for the addition or removal of 5′ phosphates are known in the art, and include without limitation enzymatic and chemical processes. Enzymes useful in the addition and/or removal of 5′ phosphates include kinases, phosphatases, and polymerases. In some embodiments, both of the two ends joined in a ligation reaction (e.g., an adapter end and a sample nucleic acid, e.g., fragmented polynucleotide duplex or single stranded polynucleotide, end) provide a 5′ phosphate, such that two covalent linkages are made in joining the two ends. In some embodiments, 3′ phosphates are removed prior to ligation.

In some embodiments, a molecular crowding agent, such as, but not limited to, polyethylene glycol, ficoll, or dextran is included in the ligation reaction mixture.

First adaptors may be incorporated separately from second adaptors, such as in a divided sample (e.g., separate ligation reaction mixtures) containing first or second sample nucleic acid molecules, or alternatively, first and second adaptors may be incorporated in temporally separated reactions in the same sample (e.g., temporally separated ligation reactions).

Single stranded adapters may be ligated to single stranded nucleic acid using methods well known in the art. For example, in a 20 μl reaction, add 1× Reaction Buffer (50 mM Tris-HCl, pH 7.5, 10 mM MgCl2, 1 mM DTT), 25% (wt/vol) PEG 8000, 1 mM hexamine cobalt chloride (optional), 1 μl (10 units) T4 RNA Ligase, 1 mM ATP with the sample nucleic acids and adapters. Incubate at 25° C. for 16 hours. The reaction is stopped by adding 40 μl 10 mM Tris-HCl pH 8.0, 2.5 mM EDTA. Similar conditions are used for ligation anchored PCR (Troutt, A. B., et al. Proc. Natl. Acad. Sci. USA. 89. 9823-9825. 1992).

### Methods for Preparing Concatenated Nucleic Acid Molecules

Methods are provided herein for preparing concatenated nucleic acid molecules. Concatenated nucleic acid molecules prepared as described herein may be sequenced or may be used in other downstream applications in which it is desirable to concatenate nucleic acid sequences together, such as, for example, in genetic analysis techniques (e.g., in microarrays), molecular cloning applications (e.g., placing functional DNA elements adjacent or within proximity of each other, for example, in a vector).

In some embodiments, the methods disclosed herein for preparing concatenated nucleic acid molecules include: hybridizing and extending first and second nucleic acid molecules; wherein the first nucleic acid molecule includes a first sample nucleic acid sequence from a subject joined to a first adaptor nucleic acid sequence that is not from the subject, and wherein the first adaptor includes a first 3′ adaptor nucleic acid sequence that includes a first extendible 3′ end; wherein the second nucleic acid molecule includes a second sample nucleic acid sequence from a subject and a second adaptor nucleic acid sequence that is not from the subject; and wherein the second adaptor includes a second 3′ adaptor nucleic acid sequence that includes a second extendible 3′ end; and wherein the first and second extendible 3′ adaptor nucleic acid sequences are capable of hybridizing (e.g., are complementary) to each other. The hybridized extendible 3′ adaptor nucleic acid sequences are extended to produce concatenated nucleic acid molecules as described herein. The concatenated extension products include: (i) at least one first nucleic acid sequence and the complement of at least one second nucleic acid sequence, separated by adaptor sequences; and (ii) at least one second nucleic acid sequence and the complement of at least one first nucleic acid sequence, separated by adaptor sequences.

In some embodiments, the methods include: (a) incorporating a first adaptor into at least one first nucleic acid molecule that includes a first nucleic acid sequence and incorporating a second adaptor into at least one second nucleic acid molecule that includes a second nucleic acid sequence, wherein the first adaptor includes a first 3′ adaptor nucleic acid sequence that includes a first extendible 3′ end and the second adaptor includes a second 3′ adaptor nucleic acid sequence that includes a second extendible 3′ end, wherein the first and second 3′ adaptor nucleic acid sequences are capable of hybridizing (e.g., are complementary) to each other; and (b) hybridizing and extending the first and second extendible 3′ adaptor nucleic acid sequences, thereby producing extension products that include concatenated nucleic acid molecules. The extension products include: (i) at least one first nucleic acid sequence and the complement of at least one second nucleic acid sequence, separated by adaptor sequences; and (ii) at least one second nucleic acid sequence and the complement of at least one first nucleic acid sequence, separated by adaptor sequences.

In some embodiments, the concatenated nucleic acid molecules include greater than two concatenated nucleic acid sequences. In some embodiments, the at least one first nucleic acid sequence includes a plurality of different first nucleic acid sequences, and/or the at least one second nucleic acid sequence includes a plurality of different second nucleic acid sequences. In various embodiments, the first and second nucleic acid sequences may be double stranded, single stranded, or may contain both double stranded and single stranded regions, and the adaptors may be double stranded, single stranded, or may contain both double stranded and single stranded regions (e.g., Y-shaped adaptors).

In some embodiments, first and/or second sample nucleic acid sequences are amplified prior to incorporation of adaptors. In some embodiments, first and/or second sample nucleic acid sequences to which adaptors have been joined are amplified prior to hybridization and extension to form concatenated nucleic acid molecules. In some embodiments, concatenated nucleic acid molecules, prepared as described herein, are amplified after concatenation (e.g., hybridization and extension of joined adaptor sequences), e.g., amplification of primer extension products that include concatenated nucleic acid molecules. In any of these embodiments, any suitable amplification method may be used, including, but not limited to PCR or a linear amplification method. In some embodiments, a nested, semi-nested, or hemi-nested PCR amplification method is used.

In some embodiments, the first and/or second nucleic acid sequences are enriched from a nucleic acid library, prior to incorporation of adaptors.

In some embodiments, concatenated nucleic acid molecules as described herein are rendered competent for sequencing. For example, the concatenated nucleic acid molecule may be made competent to hybridized to a flow cell, for example, by immobilization on the surface of a flow cell.

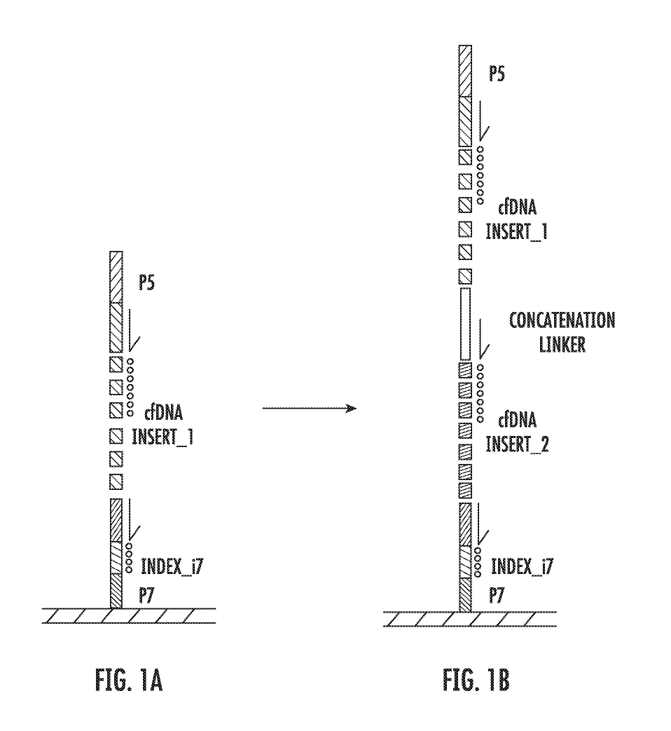

A nonlimiting embodiment of a concatenated nucleic acid molecule with two sample nucleic acid sequences separated by an adaptor sequence, prepared as described herein and immobilized on a flow cell for sequencing, is shown in FIG. 1B. For comparison, a non-concatenated nucleic acid molecule with only one sample nucleic acid sequence, is shown in FIG. 1A.

In some embodiments, a library is produced that contains a plurality of concatenated nucleic acid molecules, e.g., concatenated nucleic acid products (e.g., extension products or amplified extension products, or PCR amplicons), prepared according to any of the methods described herein.

### Sequencing

Methods for sequencing nucleic acids are provided. The methods include preparing concatenated nucleic acid molecules, employing methods described herein, and sequencing the concatenated nucleic acid products (e.g., extension products or amplified extension products, or PCR amplicons) of the methods.

In one embodiment, Illumina sequencers are used for sequencing of the concatenated nucleic acids. Illumina produces a widely used family of platforms. The technology was introduced in 2006 (www.illumina.com) and was quickly embraced by many researchers because a larger amount of data could be generated in a more cost-effective manner. Illumina sequencing is a sequencing-by-synthesis method, which differs from “454” sequencing methods, described infra, in two major ways: (1) it uses a flow cell with a field of oligo's attached, instead of a chip containing individual microwells with beads, and (2) it does not involve pyrosequencing, but rather reversible dye terminators.

In another embodiment, a dye-termination sequencing approach is used for sequencing of the concatenated nucleic acids. Dye-termination resembles the “traditional” Sanger sequencing. It is different from Sanger, however, in that the dye terminators are reversible, so they are removed after each imaging cycle to make way for the next reversible dye-terminated nucleotide. Sequencing preparation begins with lengths of DNA that have specific adaptors on either end being washed over a flow cell filled with specific oligonucleotides that hybridize to the ends of the fragments. Each fragment is then replicated to make a cluster of identical fragments. Reversible dye-terminator nucleotides are then washed over the flow cell and given time to attach; the excess nucleotides are washed away, the flow cell is imaged, and the terminators are reversed so that the process can repeat and nucleotides can continue to be added in subsequent cycles.

In another embodiment, 454 sequencing (http://www.454.com/) (e.g. as described in Margulies, M. et al., Nature 437:376-380 [2005]) is used for sequencing of the concatenated nucleic acids. The overall approach for 454 is pyrosequencing based. The sequencing preparation begins with lengths of DNA (e.g., amplicons or nebulized genomic/metagenomic DNA) that have adaptors on either end, created by using PCR primers with adaptor sequences or by ligation; these are fixed to tiny beads (ideally, one bead will have one DNA fragment) that are suspended in a water-in-oil emulsion. An emulsion PCR step is then performed to make multiple copies of each DNA fragment, resulting in a set of beads in which each one contains many cloned copies of the same DNA fragment. A fiber-optic chip filled with a field of microwells, known as a PicoTiterPlate, is then washed with the emulsion, allowing a single bead to drop into each well. The wells are also filled with a set of enzymes for the sequencing process (e.g., DNA polymerase, ATP sulfurylase, and luciferase). At this point, sequencing-by-synthesis can begin, with the addition of bases triggering pyrophosphate release, which produces flashes of light that are recorded to infer the sequence of the DNA fragments in each well as each base type (A, C, G, T) is added.

In another embodiment, the Applied Biosystems SOLiD process (http://solid.appliedbiosystems.com) is used for sequencing of the concatenated nucleic acids. The SOLiD process begins with an emulsion PCR step akin to the one used by 454, but the sequencing itself is entirely different from the previously described systems. Sequencing involves a multiround, staggered, dibase incorporation system. DNA ligase is used for incorporation, making it a “sequencing-by-ligation” approach, as opposed to the “sequencing-by-synthesis” approaches mentioned previously. Mardis (Mardis E R., Next-generation DNA sequencing methods, Annu Rev Genomics Hum Genet 2008; 9:387-402) provides a thorough overview of the complex sequencing and decoding processes involved with using this system.

In another embodiment, the Ion Torrent system (http://www.iontorrent.com/) is used for sequencing of the concatenated nucleic acids. The Ion Torrent system begins in a manner similar to 454, with a plate of microwells containing beads to which DNA fragments are attached. It differs from all of the other systems, however, in the manner in which base incorporation is detected. When a base is added to a growing DNA strand, a proton is released, which slightly alters the surrounding pH. Microdetectors sensitive to pH are associated with the wells on the plate, which is itself a semiconductor chip, and they record when these changes occur. As the different bases (A, C, G, T) are washed sequentially through, additions are recorded, allowing the sequence from each well to be inferred.

In another embodiment, the PacBio single-molecule, real-time sequencing approach (http://www.pacificbiosciences.com/) is used for sequencing of the concatenated nucleic acids. The PacBio sequencing system involves no amplification step, setting it apart from the other major next-generation sequencing systems. The sequencing is performed on a chip containing many zero-mode waveguide (ZMW) detectors. DNA polymerases are attached to the ZMW detectors and phospholinked dye-labeled nucleotide incorporation is imaged in real time as DNA strands are synthesized. PacBio's RS II C2 XL currently offers both the greatest read lengths (averaging around 4,600 bases) and the highest number of reads per run (about 47,000). The typical “paired-end” approach is not used with PacBio, since reads are typically long enough that fragments, through CCS, can be covered multiple times without having to sequence from each end independently. Multiplexing with PacBio does not involve an independent read, but rather follows the standard “in-line” barcoding model.

In another embodiment, nanopore sequencing (e.g., as described in Soni G V and Meller A., Clin Chem 53: 1996-2001 [2007]) is used for sequencing of the concatenated nucleic acids. Nanopore sequencing DNA analysis techniques are being industrially developed by a number of companies, including Oxford Nanopore Technologies (Oxford, United Kingdom), Roche, and Illumina. Nanopore sequencing is a single-molecule sequencing technology whereby a single molecule of DNA is sequenced directly as it passes through a nanopore. Nanopore sequencing is an example of direct nucleotide interrogation sequencing, whereby the sequencing process directly detects the bases of a nucleic acid strand as the strand passes through a detector. A nanopore is a small hole, of the order of 1 nanometer in diameter Immersion of a nanopore in a conducting fluid and application of a potential (voltage) across it results in a slight electrical current due to conduction of ions through the nanopore. The amount of current which flows is sensitive to the size and shape of the nanopore. As a DNA molecule passes through a nanopore, each nucleotide on the DNA molecule obstructs the nanopore to a different degree, changing the magnitude of the current through the nanopore in different degrees. Thus, this change in the current as the DNA molecule passes through the nanopore represents a reading of the DNA sequence. Another example of direct nucleotide interrogation sequencing that may be used in conjunction with the present methods is that of Halcyon.

### EXEMPLARY EMBODIMENTS

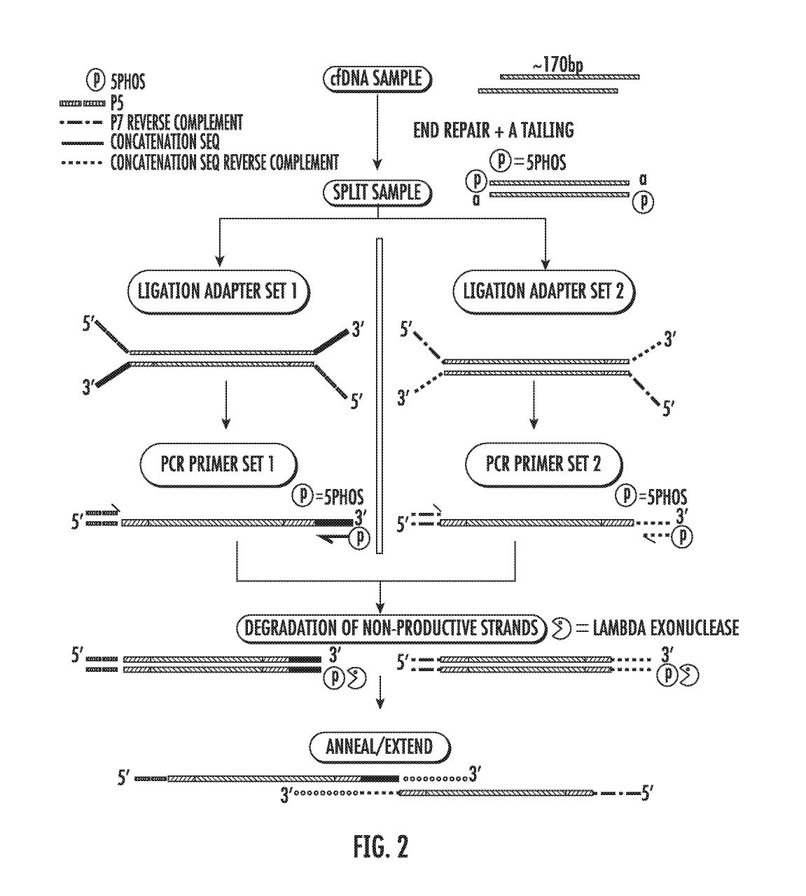

FIG. 2 shows an example of a workflow for preparation of concatenated nucleic acid sequences using a method as described herein. In the workflow shown schematically in FIG. 2, a nucleic acid sample (e.g., a cfDNA sample) is split into two samples (i.e., a “first nucleic acid molecule” sample and a “second nucleic acid molecule” sample). First adaptors are ligated to first nucleic acid molecules and second adaptors are ligated to second nucleic acid molecules. In the embodiment depicted in FIG. 2, the adaptors are ligated in separate reactions (e.g., in parallel). In an alternative embodiment, the ligation events could be temporally separated, in an undivided sample.

After ligation of the adaptors to the ends of the double stranded nucleic acid molecules, adaptor ligated nucleic acid molecules are amplified using primer sequences that are complementary to 5′ and 3′ sequences from the adaptors. Primers that are complementary to the 3′ sequences from the adaptors include a 5′ phosphate, which enables degradation of “non-productive” second strands (nucleic acid strands that not include 3′ end sequences that will hybridize for extension to produce concatenated nucleic acid sequences), for example, by an exonuclease enzyme, such as, but not limited to, lambda exonuclease. The remaining, non-degraded nucleic acid first strands anneal and are extended from extendible 3′ ends to produce concatenated nucleic acid molecules. The complementary sequences at the 3′ ends of the amplified first and second adaptors anneal under appropriate conditions and are extended to produce concatenated nucleic acid extension products that include (from 5′ to 3′) a 5′ adaptor sequence, an amplified copy of the first strand of the first nucleic acid sequence, adaptor sequences, an amplified copy of the complement of the first strand of the second nucleic acid sequence, and a 3′ adaptor sequence, and concatenated nucleic acid extension products that include (from 5′ to 3′) a 5′ adaptor sequence, an amplified copy of the first strand of the second nucleic acid sequence, adaptor sequences, an amplified copy of the complement of the first strand of the first nucleic acid sequence, and a 3′ adaptor sequence. Optionally, the extension products may be amplified prior to use in a downstream application, such as nucleic acid sequencing.

Another example of a workflow is shown in FIG. 3. In the example depicted in FIG. 3, adaptor sequences are incorporated via PCR amplification, producing PCR amplicons. Forward and reverse tailed primers that hybridize to first and second strands of nucleic acid duplex sequences of interest are used for PCR amplification. The tail sequences of the reverse primers include sequences that are complementary and include 5′ phosphate groups. After amplification, “non-productive” second strands (nucleic acid strands that not include 3′ end sequences that will hybridize for extension to produce concatenated nucleic acid sequences) are degraded, e.g., by an exonuclease enzyme, such as, but not limited to, lambda exonuclease. The complementary sequences at the 3′ ends of the amplified, non-degraded nucleic acid first strands anneal under appropriate conditions and are extended to produce concatenated nucleic acid extension products that include (from 5′ to 3′) a 5′ adaptor sequence, an amplified copy of the first strand of the first nucleic acid sequence of interest, adaptor (i.e., complement of first reverse primer tail) sequences, an amplified copy of the complement of the first strand of the second nucleic acid sequence of interest, and a 3′ adaptor sequence, and concatenated nucleic acid extension products that include (from 5′ to 3′) a 5′ adaptor sequence, an amplified copy of the first strand of the second nucleic acid sequence of interest, adaptor sequences (i.e., complement of second primer tail) sequences, an amplified copy of the complement of the first strand of the first nucleic acid sequence of interest, and a 3′ adaptor sequence. Optionally, the extension products may be amplified prior to use in a downstream application, such as nucleic acid sequencing.

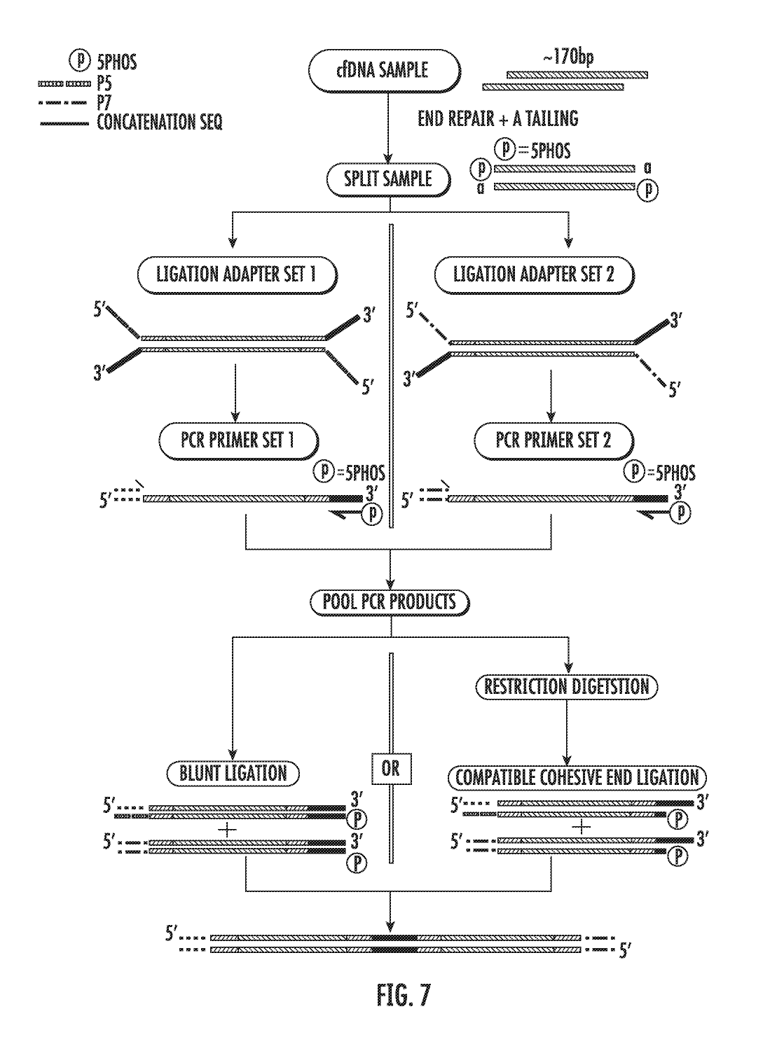

In another example, depicted schematically in FIG. 7, the sample nucleic acid molecules are concatenated via ligation. A nucleic acid sample (e.g., a cfDNA sample) is split into two samples (i.e., a “first nucleic acid molecule” sample and a “second nucleic acid molecule” sample). First adaptors are ligated to first nucleic acid molecules and second adaptors are ligated to second nucleic acid molecules. In the embodiment depicted in FIG. 7, the adaptors are ligated in separate reactions (e.g., in parallel). In an alternative embodiment, the ligation events could be temporally separated, in an undivided sample.

After ligation of the adaptors to the ends of the double stranded nucleic acid molecules, adaptor ligated nucleic acid molecules are amplified using primer sequences that are complementary to 5′ and 3′ sequences from the adaptors, thereby producing first and second amplification products from first and second adaptor ligated sample nucleic acid molecules, respectively. Primers that are complementary to the 3′ sequences from the adaptors include a 5′ phosphate, which facilitates ligation with a ligase enzyme. In one embodiment, the adaptor sequences include a restriction endonuclease recognition sequence used to create cohesive compatible ends following digestion with a restriction endonuclease. The first and second amplification products are pooled and then ligated (e.g., with a ligase enzyme), either by ligating blunt ends or by ligating cohesive compatible ends produced by digestion with a restriction enzyme, to produce concatenated nucleic acid molecules.

In one embodiment, the amplified 3′ adaptor nucleic acid sequences with extendible 3′ ends and their complements are joined via a blunt end ligation. In another embodiment, the amplified 3′ adaptor nucleic acid sequences with extendible 3′ ends and their complements include a restriction endonuclease recognition sequence and are digested with the restriction enzyme to produce cohesive ends, which are hybridized and ligated (e.g., with a ligase enzyme).

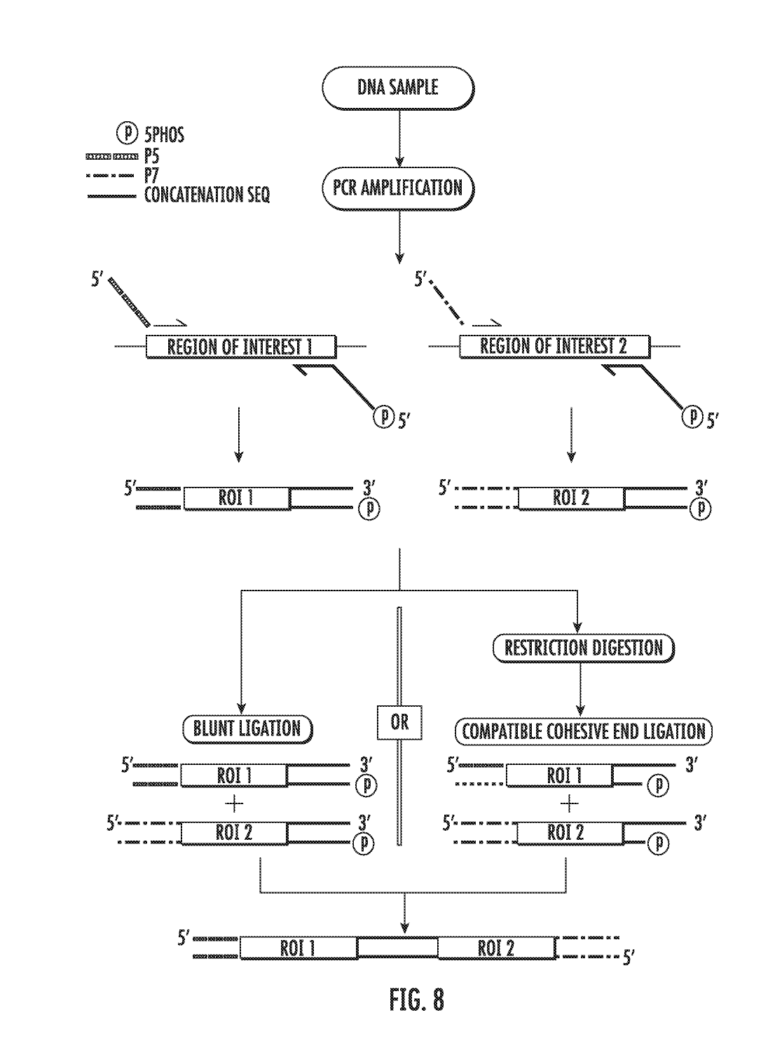

In another example, depicted schematically in FIG. 8, the sample nucleic acid molecules are concatenated via ligation. Adaptor sequences are incorporated via PCR amplification, producing PCR amplicons. Forward and reverse tailed primers that hybridize to first and second strands of nucleic acid duplex sequences of interest are used for PCR amplification. The tail sequences of the reverse primers include sequences that are complementary and include 5′ phosphate groups, which facilitates ligation with a ligase enzyme.

In one embodiment, the incorporated adaptor nucleic acid sequences are joined via a blunt end ligation (e.g., with a ligase enzyme). In another embodiment, the incorporated nucleic acid sequences include a restriction endonuclease recognition sequence and are digested with the restriction enzyme to produce compatible cohesive ends, which are hybridized and ligated (e.g., with a ligase enzyme).

The following examples are intended to illustrate, but not limit, the invention.

### EXAMPLES

### Example 1

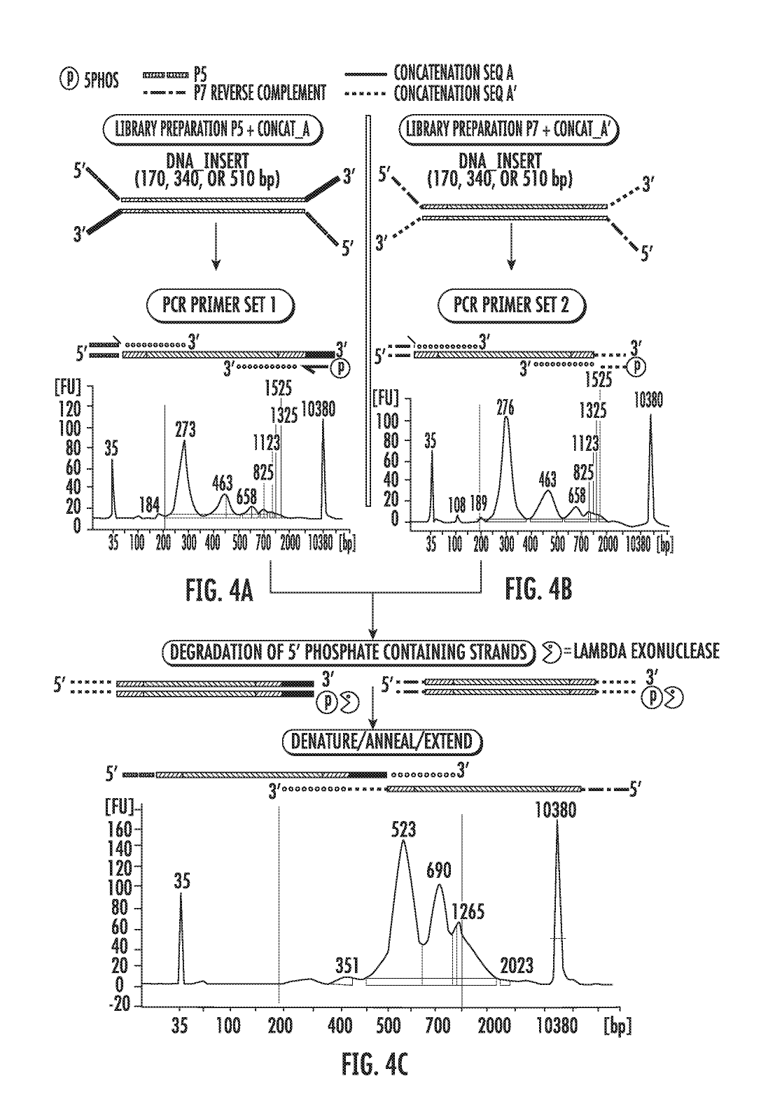

Circulating free DNA (cfDNA) was extracted from pregnant maternal plasma and subjected to a library preparation wherein multiple cfDNA fragments were concatenated together and flanked by sequencing adapters as shown in FIGS. 4A-4C, hereafter referred to as “concat_seq”. Briefly, each cfDNA sample was end-repaired and A-tailed using standard NGS library preparation chemistry, after which each sample was split into two distinct adapter ligation reactions. In one reaction, Y-shaped adapters including a P5 sequencing adapter and concatenation sequence A were ligated to the A-tailed cfDNA (FIG. 4A). In a second, separate reaction, Y-shaped adapters including the reverse complement of a P7 sequencing adapter and the reverse complement of concatenation sequence A (referred to as A′) were ligated to the A-tailed cfDNA (FIG. 4B). The PCR primers designed to hybridize to concatenation sequences A and A′ contained 5′ phosphate modifications. After exonuclease degradation, remaining PCR product was then denatured, slow cooled to anneal the concatenation sequences, and finally extended with a DNA polymerase to create a library of nucleic acid molecules consisting of two cfDNA fragments separated by the concatenation sequence and flanked by P5 and P7 sequencing adapters (FIG. 4C). The electropherograms in FIGS. 4A-4C show the ability to produce the library products as described. cfDNA has a characteristic size distribution, typically with sizes with a periodicity of 170 bp, thus leading the pattern shown in the electropherograms.

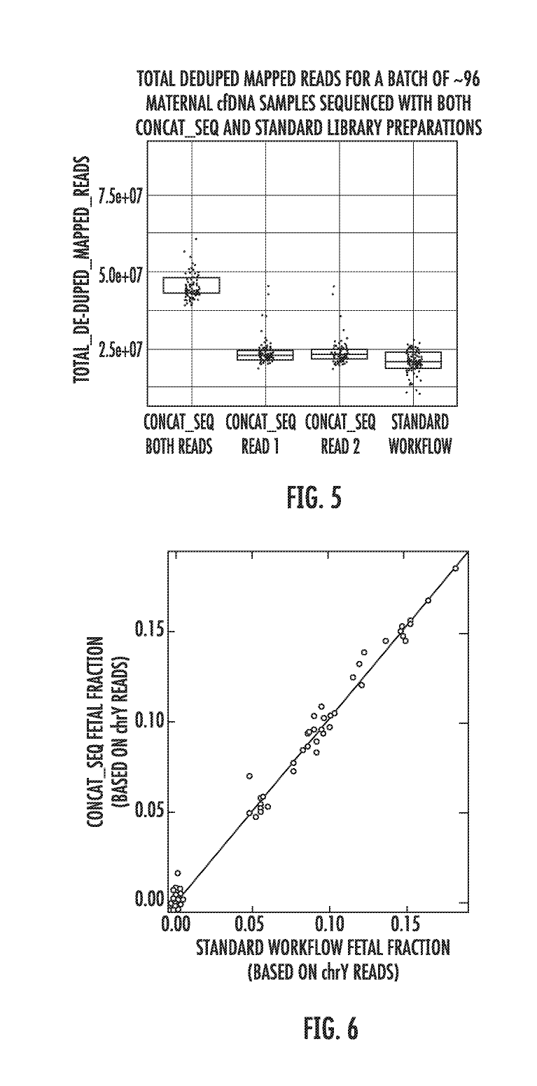

Next, replicate batches of ˜96 maternal cfDNA samples were prepared using both concat_seq library preparation described above, as well as a “standard” library preparation in which the nucleic acid molecules consisted of only one cfDNA insert flanked by P5/P7 sequencing adapters. Both groups of sample libraries were sequenced on a HiSeq 4000. Concat_seq libraries were sequenced to obtain two reads for each library (read 1 and read 2), corresponding to cfDNA insert 1 and 2. “Standard” libraries were sequenced such that only a single read was obtained, since only one cfDNA insert is present in these libraries. Importantly, each sequencing run was performed with an identical set of sequencing reagents, having equivalent costs. FIG. 5 shows the total number of mapped reads following removal of molecular duplicates, i.e., only molecules with unique genomic start positions. Approximately twice as many unique molecular reads were observed for concat_seq samples as compared to samples prepared with the “standard” workflow (˜40M mean mapped reads per concat_seq sample vs. ˜20M mean mapped reads per “standard” sample). Also, equivalent number of reads were observed from both read 1 and read 2 from the concat_seq library and each of these was roughly equivalent to the mean number of de-duped mapped reads obtained using the “standard” workflow (˜20M mean mapped reads for each).

Finally, a comparison was made to determine whether the proportion of fetal DNA reads (the fetal fraction) was equivalent between replicate samples (same 96 as above) prepared with the “standard” library preparation and the concat_seq library preparation. To do so, the proportion of Y reads present in maternal cfDNA samples harboring male fetuses was calculated. As shown in FIG. 6, approximately half of the samples harbored Y chr reads that ranged in fetal fraction from ˜4% to ˜18%. Further, the fetal fraction obtained using concat_seq library prep was equivalent to the fetal fraction obtained using the “standard” library prep, indicating that the concat_seq library preparation did not change the fundamental composition and representation of the sequenced DNA molecules relative to the “standard” library preparation.

All publications, patents, and patent applications cited herein are hereby incorporated by reference in their entireties for all purposes and to the same extent as if each individual publication, patent, or patent application were specifically and individually indicated to be so incorporated by reference.

We claim:

1. A method for preparing concatenated nucleic acid molecules, comprising:
(a) incorporating a first adaptor into at least one first nucleic acid molecule that comprises a first nucleic acid sequence and incorporating a second adaptor into at least one second nucleic acid molecule that comprises a second nucleic acid sequence, wherein the first adaptor comprises a first 3′ adaptor nucleic acid sequence comprising a first extendible 3′ end and the second adaptor comprises a second 3′ adaptor nucleic acid sequence comprising a second extendible 3′ end, wherein the first and second 3′ adaptor nucleic acid sequences are capable of hybridizing to each other; and
(b) hybridizing and extending the first and second 3′ adaptor nucleic acid sequences, thereby producing extension products that comprise concatenated nucleic acid molecules comprising at least one first nucleic acid sequence and at least one second nucleic acid sequence, separated by adaptor sequences.

2. A method according to claim 1, wherein said first and second nucleic acid sequences comprise double stranded nucleic acids with first and second ends, and wherein each of said first and second adaptors comprises: (i) a double stranded region; (ii) a single stranded nucleic acid sequence comprising an extendible 3′ end; and (iii) a single stranded nucleic acid sequence comprising a 5′ end,
wherein said first adaptor is attached to first and second ends of the first double stranded nucleic acid and said second adaptor is attached to first and second ends of the second double stranded nucleic acid, and
wherein the 3′ single stranded nucleic acid sequences of the first and second adaptors are capable of hybridizing to each other.

3. A method according to claim 2, wherein the concatenated nucleic acid molecules comprise greater than two concatenated nucleic acid sequences.

4. A method according to claim 2, wherein the 5′ single stranded sequence of the first and/or the second adaptor comprises one or more sample index sequence(s).

5. A method according to claim 2, wherein the 5′ single stranded sequence of the first and/or the second adaptor comprises a flow cell binding sequence at its 5′ end.

6. A method according to claim 1, wherein the first and second nucleic acid sequences are single stranded, wherein the first and second adaptors are single stranded, and wherein the 3′ single stranded nucleic acid sequences of the first and second adaptors are capable of hybridizing to each other.

7. A method according to claim 6, comprising addition of a 5′ phosphate group to the first and second adaptors prior to (a).

8. A method according to claim 6, wherein one or more sample index sequence and/or a flow cell binding sequence is incorporated into the 5′ end of the first and/or second nucleic acid molecule.

9. A method according to claim 1, wherein the first and second nucleic acid sequences are amplified prior to (a) or prior to (b).

10. A method according to claim 1, wherein said incorporating in (a) comprises ligation of said first adaptor to said at least one first nucleic acid sequence and ligation of said second adaptor to said at least one second nucleic acid sequence.

11. A method according to claim 10, wherein the first adaptors are ligated to the first nucleic acid sequences in a separate reaction mixture from ligation of the second adaptors to the second nucleic acid sequences.

12. A method according to claim 10, wherein the first adaptors are ligated to the first nucleic acid sequences in the same reaction mixture as ligation of the second adaptors to the second nucleic acid sequences, wherein ligation of the first adaptors is temporally separated from ligation of the second adaptors.

13. A method according to claim 10, wherein (a) comprises a ligation reaction mixture that comprises a macromolecular crowding agent.

14. A method according to claim 13, wherein the macromolecular crowding agent comprises polyethylene glycol.

15. A method according to claim 14, further comprising:
amplifying the ligated nucleic acid molecules, prior to (b).

16. A method according to claim 1, wherein said incorporating in (a) comprises an amplification reaction.

17. A method according to claim 16, wherein said amplification reaction comprises a polymerase chain reaction (PCR) reaction, wherein said first and second nucleic acid molecules are PCR amplicons.

18. A method according to claim 1, wherein said at least one first nucleic acid molecule comprises a plurality of different first nucleic acid sequences and said at least one second nucleic acid molecule comprises a plurality of different second nucleic acid sequences.

19. A method according to claim 1, wherein the first and/or second adaptors comprise a sample or source specific barcode sequence.

20. A method according to claim 1, further comprising:
(c) amplifying the extension products produced in (b).

21. A method according to claim 9, wherein said amplification of the first and/or second nucleic acid molecules comprises primers that comprise a sample or source specific barcode sequence, thereby incorporating the barcode sequence into the amplified first and/or second nucleic acid molecules.

22. A method according to claim 20, wherein said amplification of the extension products comprises primers that comprise a sample or source specific barcode sequence, thereby incorporating the barcode sequence into the amplified extension products.

23. A method according to claim 1, wherein the first and second nucleic acid molecules comprise cell-free DNA.

24. A method according to claim 23, wherein the cell-free DNA comprises cell-free tumor DNA or cell-free fetal DNA.

25. A method according to claim 1, wherein the first and second nucleic acid molecules comprise RNA or cDNA.

26. A method according to claim 1, wherein the first and second nucleic acid molecules are enriched from a nucleic acid library.

27. A method according to claim 1, wherein the extension products are rendered competent for sequencing.

28. A method according to claim 27, wherein the extension products are made competent to hybridize to a flow cell.

29. A method according to claim 28, further comprising immobilizing the extension products on the surface of a flow cell.

30. A method for nucleic acid sequencing, comprising preparing concatenated nucleic acid molecules according to claim 1, and sequencing the extension products or amplified extension products.

31. A method according to claim 30, comprising sequencing the first and second nucleic acid sequences or complements thereof in the extension products using primers that are complementary to adaptor sequences that are upstream of nucleic acid sequences in the extension product.

32. A method according to claim 30, wherein the adaptors comprise one or more sample index sequence, wherein the method further comprises sequencing at least one sample index sequence from an adaptor using a primer that is complementary to an adaptor sequence that is upstream of the sample index sequence.

33. A method according claim 30,
wherein an adaptor comprises a flow cell binding sequence at its 5′ end, and
wherein the extension products or amplified extension products are immobilized on the surface of a flow cell by hybridization of the flow cell binding sequences to complementary sequences on the flow cell.

34. A nucleic acid sequencing library, comprising a plurality of amplified extension products produced according to claim 20.

35. A method for preparing concatenated nucleic acid molecules, comprising:
hybridizing and extending first and second nucleic acid molecules,
wherein the first nucleic acid molecule comprises a first test nucleic acid sequence from a subject and a first adaptor that is not from the subject, and wherein the first adaptor comprises a first 3′ adaptor nucleic acid sequence comprising a first extendible 3′ end,
wherein the second nucleic acid molecule comprises a second test nucleic acid sequence from a subject and a second adaptor that is not from the subject, and wherein the second adaptor comprises a second 3′ adaptor nucleic acid sequence and comprising a second extendible 3′ end, and
wherein the first and second 3′ adaptor nucleic acid sequences are capable of hybridizing to each other.

36. A method for preparing concatenated nucleic acid molecules, comprising:
(a) ligating a first adaptor to at least one first double stranded nucleic acid molecule comprising first and second ends, and ligating a second adaptor to at least one second double stranded nucleic acid molecule comprising first and second ends, thereby producing first and second adaptor ligated nucleic acid molecules,
wherein each of said first and second adaptors comprises a double stranded region,
wherein said first adaptor is attached to first and second ends of the first double stranded nucleic acid molecule and said second adaptor is attached to first and second ends of the second double stranded nucleic acid molecule;
(b) amplifying the first and second adaptor ligated nucleic acid molecules in separate reaction mixtures with first and second amplification primers, thereby producing first and second amplified adaptor ligated nucleic acid molecules,
wherein one or both of the first and second amplification primers comprises a terminal 5′ phosphate group or wherein a 5′ terminal phosphate group is added to one or both ends of the amplified adaptor ligated nucleic acid molecules;
(c) combining the first and second amplified adaptor ligated nucleic acid molecules; and
(d) ligating the first and second amplified adaptor ligated nucleic acid molecules, thereby producing concatenated nucleic acid molecules.

37. A method for preparing concatenated nucleic acid molecules, comprising:
(a) incorporating a first adaptor into at least one first nucleic acid molecule that comprises a first nucleic acid sequence, and incorporating a second adaptor into at least one second nucleic acid molecule that comprises a second nucleic acid sequence,
wherein said incorporating comprises amplification, thereby producing first and second amplification products,
wherein the first nucleic acid molecule is amplified with primers that hybridize to the first nucleic acid sequence, thereby producing said first amplification product, and wherein one or both of the primers comprise a terminal 5′ phosphate group or wherein a 5′ terminal phosphate group is added to one or both ends of the first amplification product; and
wherein the second nucleic acid molecule is amplified with primers that hybridize to the second nucleic acid sequence, thereby producing said second amplification product, and wherein one or both of the primers comprises a 5′ sequence comprising a 5′ terminal phosphate group or wherein a 5′ terminal phosphate group is added to one or both ends of the second amplification product;
(b) combining the first and second amplification products; and
(c) ligating the first and second amplification products, thereby producing concatenated nucleic acid molecules.
